(19) United States
(12) Patent Application Publication
(10) Pub. No.: US 2013/0344167 A1
(43) Pub. Date: Dec. 26, 2013

(54) Galenic Composition Suitable for Administration to a Non-Human Animal, Uses Thereof, and Associated Methods

(71) Applicants: Laurent Chery, Chateaubernard (FR); Jean-Pierre Wajda-Dubos, Saint Laurent De Cognac (FR)

(72) Inventors: Laurent Chery, Chateaubernard (FR); Jean-Pierre Wajda-Dubos, Saint Laurent De Cognac (FR)

(73) Assignee: VETALIS SARL, Gente (FR)

(21) Appl. No.: 13/990,194

(22) PCT Filed: Nov. 27, 2011
(86) PCT No.: PCT/IB2011/055316

§ 371 Date: Sep. 12, 2013

(30) Foreign Application Priority Data
Nov. 29, 2010 (FR) 1004635

(51) Int. Cl. A61K 33/34; A61K 31/4015; A61K 33/30; A61K 31/375; A61K 33/24; A61K 33/32; A61K 31/30; A61K 33/06

(52) U.S. Cl. CPC A61K 33/34; A61K 31/30; A61K 31/4015; A61K 33/06; A61K 31/375; A61K 33/24; A61K 33/32; A61K 33/30
USPC 424/634; 514/499; 514/423; 424/682; 514/474; 424/637; 424/646; 424/635; 424/639; 424/643

(57) ABSTRACT

The present invention relates to a galenic composition suitable for administration to a non-human animal, including at least the following three components: one or more active principles; an agent for accelerating delitescence; an agent for delaying delitescence, wherein the agent for accelerating delitescence an the agent for delaying delitescence are integrated in the composition so as to form a matrix with the controlled release of the active principle(s). The invention also relates to the uses of said compositions, as well as to a method for measuring the in vitro delitescence thereof.

The present invention relates to galenical compositions suitable for administration to a nonhuman animal, both monogastric animals and ruminants.

In general, galenical compositions suitable for administration to animals already exist in various galenical forms, among which we may notably mention tablets, boli, pellets, granules, injections, or even supply of compositions intended to be dispersed in their drinking water, etc. In ruminants such as cows or bulls, for example, galenical compositions are often administered in the form of a bolus, introduced into the animal's rumen, where the composition can disintegrate under the action of mechanical friction with the walls of the rumen and foodstuffs, digestive juices and the native microbial flora. When the animal is monogastric, for example in the case of pigs, they can be made to ingest the galenical compositions by adding the composition, in the form of tablets for example, to their feed or adding it to their drinking water, or administered directly.

One of the main problems with all these various galenical forms is the difficulty of controlling, or of predicting with a degree of certainty, the time taken for decomposition, salting out or disintegration of the galenical composition once ingested. To date, taking into account the variability of the ingredients and active principles contained in these galenical compositions, this has not been satisfactory, or else only in a very specific area or only with a certain type of animal. Moreover, until now it has not been possible to determine, in advance, the life-time of the composition once inside the animal's stomach or rumen. Now, this is important from the economic standpoint and from the standpoint of the animal's general health, because with the existing solutions the animals receive either too much, or not enough of the active principles, as we are unable to know with certainty when the previous administration will have stopped producing its effects.

However, the present applicant has succeeded in developing a galenical composition with which these various problems can be overcome, and in doing so, has discovered other interesting properties and uses of the compositions as well as certain active principles that are integrated therein. These various objects will be identified in the detailed description of the invention, which will be given below.

Firstly, the applicant was able to develop a galenical composition suitable for administration to an animal, preferably a farm animal, comprising at least the following three components:
one or more active principles;
a disintegration accelerating agent;
a disintegration delaying agent;
   and in said composition the agent for controlling disintegration is different in its function from the disintegration delaying agent and the two are integrated in the composition so as to form a matrix with controlled release of the active principle or principles.

“One or more active principles” means one or more substances capable of having a beneficial effect on the physiology or biological state of the farm animal. In this way, and according to the present invention, various active principles can be integrated therein. A discussion of the latter will be presented below.

For example, and preferably, trace elements will be selected, in the form of salts that are water-soluble or water-dispersible, or even very sparingly soluble, or almost insoluble, of an organic or mineral nature based on copper, zinc, iodine, cobalt, manganese, iron, selenium and molybdenum.

Thus, those will be preferred that are selected from the group consisting of ferrous carbonate, ferrous chloride tetrahydrate, ferric chloride hexahydrate, ferrous citrate hexahydrate, ferrous fumarate, ferrous lactate tetrahydrate, ferric oxide, ferrous sulfate monohydrate, ferrous sulfate heptahydrate, ferrous chelate of amino acids, hydrate, iron chelate of glycine, hydrate, iron pidolate, calcium iodate hexahydrate, anhydrous calcium iodate, sodium iodide, potassium iodide, cobalt acetate tetrahydrate, basic cobalt carbonate monohydrate, cobalt carbonate hexahydrate, cobalt chloride hexahydrate, cobalt sulfate heptahydrate, cobalt sulfate monohydrate, cobalt nitrate hexahydrate, cobalt pidolate, cupric acetate monohydrate, basic copper carbonate monohydrate, cupric chloride dihydrate, copper methionate, cupric oxide, cupric sulfate pentahydrate, cuprous chelate of amino acids, hydrate, cuprous chelate of glycine, hydrate, copper chelate of the hydroxy analog of methionine, copper pidolate, manganous carbonate, manganous chloride tetrahydrate, manganese acid phosphate trihydrate, manganous oxide, manganic oxide, manganous sulfate tetrahydrate, manganous sulfate monohydrate, manganese chelate of amino acids, hydrate, manganese chelate of glycine, hydrate, manganese chelate of the hydroxy analog of methionine, manganese pidolate, zinc lactate trihydrate, zinc acetate dihydrate, zinc carbonate, zinc chloride monohydrate, zinc oxide, zinc sulfate heptahydrate, zinc sulfate monohydrate, zinc chelate of amino acids, hydrate, zinc chelate of glycine, hydrate, zinc chelate of the hydroxy analog of methionine, zinc pidolate, ammonium molybdate, sodium molybdate, sodium selenite, sodium selenate, the organic form of selenium produced by Saccharomyces cerevisiae, selenomethionine (inactivated selenium yeast), and the selenomethionine produced by Saccharomyces cerevisiae (inactivated selenium yeast).

Moreover, it may be advantageous to include, in the galenical compositions according to the invention, a composition or a mixture based on one or more vitamins, provitamins, or salts thereof, perfectly water-soluble, water-dispersible, or very sparingly soluble or even insoluble, for example fat-soluble vitamins that are normally water-insoluble but are made water-dispersible after adsorption and/or encapsulation on a support prior to their integration in the dosage form, but also water-soluble vitamins, protected by encapsulation in a matrix of a hydrophobic nature, in order to delay their escape in the surroundings. With regard to vitamins, they are preferably in the form of powders that can easily be mixed together: whether the vitamins are of water-soluble or fat-soluble nature, after for example encapsulation and/or adsorption, either in a matrix or else on a suitable pulverulent support.

Thus, the following will be preferred as vitamin: vitamin A, vitamin D2 (ergocalciferol), 25-hydroxycalciferol, vitamin D3 (cholecalciferol), beta-carotene (provitamin A), vitamin E, vitamin K, for example in the form of menadione bisulfite, vitamin B1, for example in the form of thiamine hydrochloride and/or thiamine mononitrate, vitamin B2, for example in the form of riboflavin and/or riboflavin phosphate (monosodium ester salt), vitamin B6, for example in the form of pyridoxine hydrochloride, vitamin B12 in the form of cyanocobalamin, vitamin C in the form of L-ascorbic acid, sodium L-ascorbate, calcium L-ascorbate, palmityl-6-L-ascorbic acid, calcium salts, sodium ascorbyl monophosphate, pantothenic acid for example in the form of calcium D-pantothenate, or D-pantothenol, vitamin PP, for example in the form of nicotinic acid, niacin, and/or nicotinamide-niacinamide, vitamin B9, for example in the form of folic acid, vitamin H2, B7 or BW, in the form of biotin, choline, for example in the form of choline chloride, choline dihydrogen citrate, choline bitartrate, inositol, carnitine, for example in the form of L-carnitine, L-carnitine-L-tartrate, betaine, for example in the form of anhydrous betaine, betaine monohydrate, betaine hydrochloride, taurine, and similar.

Among other desirable active principles, we may also mention the macroelements, which are generally based on one or more minerals containing calcium, magnesium, sodium and/or phosphorus, potassium or sulfur. The latter are preferably in the form of powder and correspond to mineral salts either of organic nature or of completely mineral nature, i.e. inorganic salts, and have a solubility in water above 0.25 g in 100 g of water. Preferably, the apparent density of these powders is above 0.45 and they have a granulometry below 800 microns. Preference will then be given to salts that are soluble and have high bioavailability, as the macroelements have be supplied in large amounts generally in the form of immediate supply for relatively short periods, of the order of a few days at most. Preferably, the macroelement is selected from the group consisting of the calcium and magnesium L-pidolates, the calcium and magnesium chelates of amino acids, the calcium or magnesium glycinates, or any other calcium or magnesium complex or chelate containing a ligand of an organic nature in its structure, the calcium and magnesium lactates, the calcium and magnesium gluconates, the calcium and magnesium formates, the calcium and magnesium citrates, magnesium sulfate and calcium sulfate.

The galenical compositions according to the present invention can also contain other active principles, notably one or more prebiotics in powder form. In general the expression “prebiotic”, when used in the context of the present invention, is to be understood as denoting an oligosaccharide or a polysaccharide, which performs the role of substrate for promoting growth of certain colon bacteria, and notably the lactobacilli and bifidobacteria. When absorbed by the oral route as dietary supplement or food additives, the prebiotics are not digested in the digestive system and by the intestine of farm animals, but they pass directly into the colon where they perform a targeted role, so that they only promote the beneficial bacteria of the intestinal flora.

Thus, and according to the present invention, it is preferable that the prebiotic or prebiotics are selected from the group consisting of fructo-oligosaccharides (FOS), inulin and/or inulin derivatives, manno-oligosaccharides (MOS) and/or combinations of MOS and of glucose (β-glucans), for example MOS 500, which is a specific combination of manno-oligosaccharides and of β-glucans, obtained by natural extraction from the cell wall of Saccharomyces cerevisiae yeasts.

Another possibility of preferred active principle consists of the probiotics. The expression “probiotic”, when used in the context of the present invention, denotes live microorganisms, bacteria or yeasts, naturally present in the body, notably in the intestinal flora. Their absorption by the oral route, in the form of food additives or directly in foodstuffs, stimulates the growth of useful bacteria to have a beneficial effect on health. They contribute to the digestion of fiber, reinforce the immune system, and act against diarrhea, atopic eczema, gastric ulcer, etc. These preparations of probiotics, when used in the context of the present invention, are generally encapsulated, dispersed and/or adsorbed on a solid support of the powder type, for example calcium carbonate, dextrose or sorbitol, for reasons of practical convenience. Thus, the probiotics usable according to the present invention are preferably selected from the group consisting of Saccharomyces cerevisiae, Enterococcus faecium, Bacillus cereus var. toyoi, Bacillus subtilis, Bacillus licheniformis, Bacillus amyloliquefaciens, Clostridium butyricum, Pediococcus acidilactici, Lactobacillus rhamnosus, Lactobacillus farciminis, and Kluyveromyces marxianus-fragilis.

According to a preferred embodiment of the present invention, the galenical composition also comprises one or more active principles as powder in the form of proteins, peptides, enzymes and/or free amino acids, which can be either of vegetable origin, or of animal origin, or resulting from biofermentation of microorganisms, or of synthetic origin, and in particular proteins and/or water-soluble concentrates of milk proteins as powder, resulting from the cracking of milk such as lyophilized or atomized colostrum powder, whey in the form of powder, immunoglobulins such as purified or IgG-enriched fractions, lactoferrin, lactoperoxidase, animal or vegetable enzymes, and more particularly, Promutase (SOD), 3-phytase, 6-phytase, the endo-1,4-betaglucanases, the endo-1,4-betaxylanases, or other enzymes improving or promoting an animal's digestion. When amino acids are integrated as active principle in the present composition, it is preferable to use free amino acids or those in the form of salts, or peptides, notably L-carnitine, more particularly in its dipeptide form, or any other water-soluble peptide of molecular weight greater than a dipeptide.

Advantageously, the compositions according to the present invention also comprise one or more active principles of vegetable origin, for example powders or a mixture of powders of dried, ground, and/or micronized plants, or else one or more vegetable extracts, preferably in the form of dry extracts, of vegetable active principles, for example the fraction or the purified molecules, such as saponins, polyphenols, flavonoids, alkaloids, etc., water-soluble or water-dispersible, obtained by means of a suitable extraction solvent of high or medium polarity, such as water, alcohol, or an aqueous-alcoholic or water-acetone mixture of low titer; this same group of active principles may also include one or more vegetable extracts of liquid type, for example one or more mother tinctures, previously dispersed on a suitable powder support.

In the case when these vegetable extracts would be rather fat-soluble or of a hydrophobic nature, it is preferable to make them water-dispersible by encapsulation or adsorption on a powder support such as cyclodextrins, gum arabic, silicas, maltodextrins, inulin, etc., before integration. Moreover, it is also possible to integrate one or more essential oils, containing monoterpenes and sesquiterpenes, and/or one or more purified aromatic molecules of natural origin or synthetic, preferably adsorbed, coated or encapsulated on a powder support, so as to be rendered water-soluble or water-dispersible. This same group of active principles can also include “dry” extracts of plants of lipophilic nature, such as acetone extracts or of lower polarity, or else extracts obtained by extraction with supercritical CO₂, or with some other nonpolar solvent, but rendered dispersible in water after adsorption or encapsulation on a support of powder type.

Finally, active principles included in the compositions according to the present invention can be active principles used in the case of therapeutic treatment, such as antibiotic therapy, notably one or more molecules with a therapeutic objective, such as antibacterials, antiparasitics, anthelmintics, including cestocides, nematocides, fasciolicides; tenicides, anticoccidians and anticryptosporidians, antiparamphistomes, antiprotozoa, antimycotics, notably against actynomycoses and candidoses; anti-inflammatories, antiallergics and immunomodulators, such as central analgesics, oral anti-inflammatories, and antihistamines; in the area of digestion and hepatology, anti-ulcer drugs and anti-emetics, antidiarrheics and antispasmodics, enzymes and digestive flora, laxatives, purgatives, antimeteorism agents and stimulants of ruminal motility, modifiers and regulators of hepatic function, digestive regulators of rumination; in the area of cardiology, angiology, vasodilators, active principles with a vascular target; in the immune system, active principles used in immunotherapy, supply of immunoglobulin, interferon, immunomodulators; in the area of metabolism, or nutrition, anabolic drugs, antianemic agents, antiketosis agents, oral rehydrating agents, metabolism of calcium, magnesium, and phosphorus, lipotropic factors and liver and kidney disease, vitamin therapy and oligo therapy; active principles used in hormonology, reproductive endocrinology, fertility hormones, antihyperthyroidism agents, antigalactogens, abortifacients, hormone inhibitors; in the area of the musculoskeletal system, antirheumatics, antiarthritics, stimulants of muscles and of the locomotor apparatus, antimyopathy; in the area of the respiratory system, respiratory analeptics, bronchodilators, mucoregulators and expectorants, antitussives, anti-infectious agents; in the urinary system, diuretics, urinary acidifiers, urinary antispasmodics; in the neurological system, general anesthetics, analgesics, anticonvulsants, sedatives and tranquilizers, etc.

Preferably, the active principles are present in the compositions of the present invention in amounts between 0.5 wt % and 90 wt % relative to the total weight of the composition.

The compositions according to the present invention also comprise a disintegration accelerating agent. “Disintegration accelerating agent” means one or more substances whose function is to facilitate the entry of water into the composition according to the invention, once the latter is in an aqueous medium, for example in the stomach or rumen of an animal; the applicant has moreover discovered that by selecting a certain type of disintegration accelerating agent, it is possible to accelerate disintegration as a function of the desired result; examples of suitable agents that accelerate disintegration will be given below. In fact, the applicant's preferred choice was lignosulfites or lignosulfonates as preferred agent for accelerating disintegration. The lignosulfonates are water-soluble mono- or poly-electrolyte anionic polymers obtained from the paper pulp and wood treatment industry, obtained by treatment of lignin under the action of a bisulfite acidic solution. The sulfonated lignins thus obtained are neutralized under the action of a base, then concentrated to dryness to give lignosulfonates in powder form. The lignosulfonates are generally in the form of highly water-soluble fine powders and are strongly hygroscopic, showing a tendency to form lumps and to take up moisture. The lignosulfites or lignosulfonates have a very wide range of molecular weight, being very polydispersed, and they can even repolymerize under certain conditions, with molecular weights between 10000 and 200000 dalton. The cations that attach to the sulfonic groups are generally a mixture of NH₄⁺ and Ca₂⁺—or else single ions such as Na⁺, Ca₂⁺, K⁺, and NH₄⁺. The lignosulfonates are generally known for use as colloidal agent in compositions for animal feed, or even as delaying agent for mortar compositions, but a priori not as disintegration accelerating agent as in the compositions according to the present invention.

Preferably, the applicant selected the lignosulfonates based on calcium and ammonium, or a mixture thereof, or else based on sodium, or potassium. They advantageously have a proportion of metal ions between 3 and 15%, and a total sulfur content between 7% and 7.5%, with a residual water content less than or equal to 7%. Even more preferably, the lignosulfonates used in the present invention are:
the mixed acid lignosulfonates, available for example from the company Tembec, for example the lignosulfonate ARBO C12 NH₄/Ca;
the simple acid lignosulfonates, available from the company Borregaard, under the name Borresperse AM 320 (NH₄), Lignobond DD (Ca), also available from the company Borregaard, or else Arbo T11 N5 (NH₄), available from the company Tembec;
the simple neutral lignosulfonates, such as Ultrazine Ca available from the company Borregaard;
the simple basic lignosulfonates, such as Arbo N18 (Na) from Tembec, Borresperse (Na) from Borregaard, Ultrazine Na from Borregaard and Arbo K18 (K) from Tembec.

Preferably, the disintegration accelerating agent is present in the compositions according to the present invention in amounts between 3 wt % and 25 wt % relative to the total weight of the composition, according to a preferred variant between 3% and 16%, and according to another variant between 3 wt % and 8 wt % relative to the total weight of the composition.

In addition, the applicant found that there may be, in certain cases, a fairly significant dose effect with regard to the amount of accelerating agent, notably when the active principles are, by their nature, moderately water-soluble or very sparingly water-soluble, i.e. disintegration is accelerated significantly on increasing the amount of accelerating agent in the composition. Although it is undoubtedly also present in the compositions based on active principles that are very water-soluble, demonstration of this phenomenon in this type of composition is much more difficult because the very water-soluble active principles dissolve too quickly.

As was mentioned above, the compositions according to the invention also comprise a disintegration delaying agent. “Disintegration delaying agent” means one or more substances whose function is to prevent water entering the composition, and thus delay disintegration; it should be noted that the function of the delaying agent is much different than that of the disintegration accelerating agent. The applicant discovered that the use of a fat is very suitable for this role of disintegration delaying agent. Thus, it is preferred for the disintegration delaying agent to be a fat that is solid at room temperature, preferably in the form of fine powders of granulometry less than or equal to 800 microns.

Fats are to be preferred that are of vegetable origin and/or obtained by chemical synthesis, for example catalytic hydrogenation of vegetable oils. Thus, the preferred fats in solid particulate form are selected from the group consisting of hydrogenated colza oil, hydrogenated soybean oil, hydrogenated cottonseed oil, hydrogenated palm oil, hydrogenated cabbage palm oil, hydrogenated castor oil, vegetable waxes, esters of acids and fatty alcohol, cerides, cetyl palmitate, for example obtained from spermaceti, carnauba wax, white beeswax, candelilla wax, stearin, stearic acid, glycerol trihydroxystearate, microcrystalline wax, solid paraffin, and white melamine wax.

Preferably, the disintegration delaying agent is present in the compositions according to the present invention in amounts between 1% and 12%, preferably 1% to 8%, more preferably between 2% and 10%, and even more preferably between 3 wt % and 12 wt % relative to the total weight of the composition.

Moreover, the composition can advantageously, but not necessarily, and depending on the intended use of the galenical composition, comprise a ballasting agent. “Ballasting agent” means one or more substances or mixtures of substances whose only function is to increase the density of the composition, with the objective of keeping the composition in the animal's stomach or rumen. The ballasts usable in the context of the present invention are essentially in the form of water-insoluble fine powders, preferably with a particle size below 500 microns. The ballast powders used must preferably have an apparent density greater than or equal to 2.40 g/cm³, thus fully fulfilling their role of densifying agent, which can prevent regurgitation and reflux, when the galenical composition is in the form of a bolus, from the rumen.

In fact, it is preferable that when the composition is in the form of a bolus, the latter should have a density, once the components have been compressed, above 1.5 g/cm³, more preferably above 1.8 g/cm³ or close to 2.0 g/cm³, or even more preferably above 2.0 g/cm³.

Thus, the ballasts usable in the context of the present invention are therefore preferably selected from the metals or from the metal alloys displaying such characteristics. This applies notably and preferably to iron, steel, cast iron, bronze, copper, brass, nickel, tungsten, zinc, constantan, which is an alloy of copper and nickel, chromium, manganese, ferronickel. It is also preferable for the ballasts to be free from all toxic metals, such as lead, arsenic, cadmium, and mercury, in order to be compatible with use in the context of animal health or feeding.

Among the various preferred ballasts mentioned above, the most preferred example is that of iron powder. Among the various iron powders usable in the present context, we may mention the following commercial specialties: Nutrafine RS from the company HOGANAS, MH4024 offered by this same company, the Carbonyl Iron powders from the company BASF, 42 DR ELTRO 400/30 or ATOMET 110 marketed by ECKA GRANULES, etc.

Yet another example tested in the context of the present invention is that of an atomized zinc powder, marketed by the company CASTOLIN under the trade reference ROTOTEC 29230.

The amount of ballast present in the compositions according to the present invention can vary quite widely, and thus its percentage by weight relative to the total weight of the composition is preferably between 0% and 50%. Preferably, the ballasting agent is in particulate iron form in amounts between 7 wt % and 25 wt % relative to the total weight of the composition. Even more preferably the ballasting agent is in particulate iron form in amounts between 9 wt % and 25 wt % relative to the total weight of the composition.

When desirable, for example to increase the hardness of the composition according to the invention in the form of tablets or of a bolus, it may be advantageous for a compression agent to be present in said composition according to the invention. Thus, “compression agent” means a compression aid of mineral nature, inert, which does not supply any active principle. In the context of the present invention, the applicant discovered, quite surprisingly, that in contrast to various galenical excipients and/or compression aids used traditionally in the pharmaceutical industry, which disintegrate very quickly in water after a few hours, certain raw materials of mineral origin could be used judiciously for galenical purposes as compression aids, to provide better control of disintegration on the one hand and/or prolong, in certain cases significantly, the disintegration time of the composition when it was in the form of a bolus, disintegration of which was otherwise of short duration. Thus, addition of a compression aid made it possible to increase the disintegration time from a few hours to several days, or even several weeks. As mentioned previously, these fillers are not those used conventionally in the pharmaceutical industry for compression: for example modified starches, dextrates of the EMDEX type, lactose for compression of the Flowlac type, Pharmatose, or sorbitols for compression of the NEOSORB type.

In fact, the latter have proved to be far too soluble, or facilitate dispersion in water excessively, or else have a disintegrant effect, to be useful as compression agent in the present invention.

In the same line of thought, coating or enrobing agents such as: hydroxypropylcellulose (HPC in its highly substituted version) or hydroxypropylmethylcellulose (HPMC in its highly substituted version), framed for their film-forming properties, were tested, leading to mediocre results, with at most disintegration times of a few hours.

Thus, the applicant preferred to employ mineral raw materials as compression agent, and even more preferably those based on calcium, magnesium, and/or phosphorus in the form of an inorganic salt of mineral origin. The preferred compression agent according to the present invention is therefore selected from the oxides, the hydroxides, the carbonates and the phosphates, the content or supply of which in the compositions according to the present invention does not have maximum daily allowances, in contrast to the conventional mineral salts based on trace elements, the maximum daily doses of which are stipulated in regulations. Preferably, the salts selected are in the form of fine powders with a granulometry less than or equal to 800 microns, making them compatible with operations of homogeneous mixing with the other ingredients of the composition. The compression agents adopted also have, preferably, a very low, or even almost zero, solubility in water, i.e. below 0.25 g in 100 g of water, and preferably below 0.15 g in 100 g of water. Regarding the other preferred characteristics of the compression agents according to the present invention, we may also mention an apparent powder density above 0.45, making it possible to produce a sufficiently dense bolus while reducing the amount of ballasting agent, which has the additional advantage of leaving room in the formula for the other ingredients, including the active principles and the other optional excipients. It is particularly advantageous for the compression agent to be suitable for wet granulation, as well as for compression, i.e. capacity for forming particles that are sufficiently resistant to crushing once dried. Among all the possible compression agents, the applicants prefer that those used in the invention are selected from the group consisting of magnesium oxide, quicklime, dolomite lime, slaked lime, magnesium hydroxide, anhydrous dicalcium phosphate, tricalcium phosphate, natural or precipitated calcium carbonate, high density calcium carbonate, lithothamnion, magnesium carbonate, heavy basic magnesium carbonate, or a mixture thereof, and is preferably magnesium oxide. Even more preferably, these compression agents are all used in the form of powder.

According to a particularly advantageous aspect, the compression agent is present in the composition according to the invention in amounts between 0% and 70%, preferably between 0% and 60%, more preferably between 0 wt % and 40 wt % relative to the total weight of the composition.

During the development of the compositions according to the present invention, the applicant also discovered that a certain number of parameters other than those already mentioned could have an influence on the disintegration time to varying degrees. Thus, determination of these parameters enabled him to develop another definition of the compositions according to the present invention in relation to the desired or real disintegration in vitro or in vivo of the composition, also valid, and verified by statistical modelling. One of these parameters is the solubility index, which is an absolute value obtained by adding together the relative contributions to solubility of a given component in the composition, calculated by multiplying the known solubility in grams in 100 g of water of said component by the percentage by weight of said component in the composition:

Solubility index=SUM 1 to N(Solubility Component X*% incorporation of Component X).

The solubility values of the components are those:
either available in the official documentation of the supplier of the component or its data sheets;
or obtained from reference works, such as:
   “Nuffield Book of Data”—Rev. ed.: section 5.3 (inorganic compounds: Physical and thermochemical data), pages 61 to 101;
   “CRC Handbook of Chemistry and Physics”, 88th Edition (2007-2008) by David R. Lide;
   the reference “Solubilities: inorganic and metal-organic compounds: a compilation of solubility data from the periodical literature”, Linke, William, Seidell, Atherton, ACS 1958-1965;
   “Perry's Chemical Engineers Handbook”, 7th Ed., Robert H. Perry, 1977;
   “Usuel de Chimie Générale et Minérale” [Manual of General and Mineral Chemistry], 1920-today, Bernard, M., and Busnot, F.
or calculated in the laboratory, with regard to the soluble active principles for which solubility data are not stated by the suppliers and/or available in the literature.

The protocol for solubility determination for these components was as follows:

Put a beaker containing 200 g of cold water at a temperature between 18 and 25° C. on a magnetic stirring plate. Incorporate while stirring and in very large excess, up to saturation, a given amount of the substance whose solubility is to be measured. After stirring for one hour, filter the saturated solution on a funnel provided with a filter paper (the undissolved portion being retained on the filter). Collect the filtrate. Analyze the latter using a desiccator/moisture meter of the type Sartorius MA 150: test sample=2 to 3 g of filtrate, spread and distributed uniformly on a metal dish, and heated to a temperature of 105° C., in order to measure the dry residue at constant weight, after evaporation of the water. The value of the dry residue is given by the instrument in grams in 100 g of solution. The water content is thus determined by difference. Then, by calculation, the solubility of the substance to be analyzed is deduced, expressed in grams in 100 g of water.

In fact, the applicants discovered that a high value of the solubility index leads to compositions that generally disintegrate quite quickly, whereas a low value of the solubility index tends rather to lead to compositions that disintegrate very slowly. This determination was confirmed by an analysis and statistical modelling conducted on the various compositions prepared by the applicant during development of the invention.

Thus, according to a first aspect, a galenical composition according to the invention is proposed that comprises:
one or more active principles;
a solubility index in water at room temperature between 4.5 and 38;
a component based on lignosulfonate in amounts between 3 wt % and 25 wt % relative to the total weight of the composition;
a component based on fat in amounts between 1 wt % and 8 wt % relative to the total weight of the composition,
   said composition displaying a disintegration between one hour and less than or equal to 30 days.

According to a second aspect, a galenical composition is proposed that comprises:
one or more active principles;
a solubility index in water at room temperature between 2.5 and 11.5;
a component based on lignosulfonate in amounts between 3 wt % and 16 wt % relative to the total weight of the composition;
a component based on fat in amounts between 2 wt % and 10 wt % relative to the total weight of the composition;
   said composition displaying a disintegration greater than 30 days and less than or equal to 90 days.

According to a third aspect, a galenical composition is proposed that comprises:
one or more active principles;
a solubility index between 2 and 4;
a component based on lignosulfonate, in amounts between 3 wt % and 8 wt % relative to the total weight of the composition;
a component based on fat, in amounts between 3 wt % and 12 wt % relative to the total weight of the composition;
   said composition displaying a disintegration in vivo greater than 90 days and less than or equal to 180 days.

Advantageously, the compositions thus defined can also further comprise a compression agent in amounts between 0 wt % and 70 wt % relative to the total weight of the composition, preferably between 0% and 60%, and more preferably between 0 wt % and 40 wt % relative to the total weight of the composition.

According to a fourth aspect, a galenical composition is proposed that comprises:
one or more active principles;
a solubility index in water at room temperature between 2 and 4;
a component based on lignosulfonate in amounts between 3 wt % and 8 wt % relative to the total weight of the composition;
a component based on fat in amounts between 3 wt % and 12 wt % relative to the total weight of the composition;
a compression agent, in amounts between 4 wt % and 30 wt % relative to the total weight of the composition;
   said composition displaying a disintegration greater than 180 days.

The applicant also found another phenomenon, when the compositions according to the invention comprise zinc oxide alone as active principle, namely that it is necessary to include a compression agent, preferably magnesium oxide, in the composition, otherwise the composition did not behave as intended and had a tendency to dissociate, or even break down, erratically, rather than disintegrate.

Apart from the components given above, the composition can of course comprise aids and excipients, for example binders, flavorings, sweeteners, flavor enhancers, lubricants serving as a compression aid when the composition is in the form of a bolus or tablet, and the like. Examples of these adjuvants are given below for purposes of illustration:
binders: water-soluble cellulose ethers, povidones, gum arabic, etc., in amounts between 3 wt % and 7 wt % relative to the total weight of the composition;
lubricants as compression aid: magnesium stearate, in amounts between 1 wt % and 6 wt % of the total weight of the composition;
sweeteners, flavorings, flavor enhancers, in amounts between 0.05% and 2%.

In general, the total amount of aids and excipients is between 1% and 10%.

During development of the compositions described above, the applicant made astonishing discoveries, as it was found that one of the active principles, namely calcium and/or magnesium pidolate, had remarkable effects on the animals that ingested it. In particular, calcium pidolate, as well as magnesium pidolate, optionally mixed with calcium pidolate, had a remarkable effect on nonhuman female animals that had just given birth, and producers of milk, as well as their progeny that fed on the milk produced. In fact, the applicant found notably in the cow post-partum, for example, that the latter began to produce significant amounts of milk much earlier and in larger quantities than other cows in the same situation, but only receiving a conventional calcium supply. A similar effect was found in the sow, notably in that the piglets fed by the mother that had received peri-partum calcium and/or magnesium pidolate showed a larger weight gain than a group of corresponding piglets whose feeding sow had not been supplied with these salts. It was found that farrowing was accelerated, with the consequence that the sow can produce more litters per year, and moreover less time is required for supervision owing to the shorter farrowing time.

Therefore in addition to the effect of the supply of calcium, the applicant discovered that the pidolate ion, in the form of a calcium and/or magnesium salt, could not only supply calcium to a nonhuman animal, but also induce the mobilization of endogenous calcium ions in the animal in question. This effect had never a priori been detected or demonstrated previously. When the animal is a milk-producing cow, the consequence of this effect is to stimulate resumption of production much earlier than in a cow that has not received said supply, which has in its turn the consequence of making the cow more productive in the long term in terms of the amount of milk produced. Thus, the applicant has discovered a surprising use of calcium and/or magnesium pidolate, which is very interesting from the physiological and economic standpoint, for the animal breeder. This has led to the development of other uses of calcium and/or magnesium pidolate, as indicated below.

According to one aspect, the invention preferably relates to the use of calcium and/or magnesium pidolate for supplying calcium and/or magnesium, for a nonhuman animal, preferably a farm animal. Preferably, calcium and/or magnesium pidolate is used for stimulating milk production in a nonhuman female animal, preferably postpartum. Even more preferably, the nonhuman animal is a ruminant, and even more preferably the nonhuman animal is selected from the group consisting of bovines, sheep, goats, the deer family, the camel family, and is preferably a bovine. According to another preferred use, the nonhuman animal is monogastric, preferably selected from the group consisting of pigs, the rabbit family, equines, pets, preferably dogs and cats, and even more preferably it is a pig.

It should be noted that calcium and/or magnesium pidolate can be used in any suitable composition or vehicle, for example simply by adding the active principle or a mixture of two active principles in question to the feed of the animal in question. It is, however, preferable for the calcium and/or magnesium pidolate to be used in its L-pidolate form, and preferably integrated as an active principle in a galenical composition according to the invention.

According to another aspect of the present invention, it is envisaged to use calcium and/or magnesium pidolate as a supply of macroelements for nonhuman animals, preferably farm animals. In this case, it is preferred that this supply is effected via a composition according to the invention, preferably in the form of a bolus, and that the nonhuman animal is a ruminant. As before, it is then preferable for the nonhuman animal to be selected from the group consisting of bovines, sheep, goats, the deer family, the camel family, and is preferably a bovine, when the animal in question is a ruminant. However, it can also be used for other animals, and in other forms of presentation, for example in the form of tablets, for example tablets to be crunched. In this case, it is preferable for the nonhuman animal to be selected from the group consisting of pigs, the rabbit family, equines, pets, preferably cats and dogs, and is preferably a porcine animal.

According to another aspect of the invention, it is envisaged to use calcium and/or magnesium pidolate for stimulating milk production in nonhuman mammals postpartum, preferably farm animals. Preferably, said animal is a cow, although other nonhuman animals could be considered. In this case, it is preferable for the pidolate in question to be integrated in a composition according to the invention, and more preferably in tablet form and for the animal to be a sow. The calcium and/or magnesium pidolate can then be administered as it is, but will preferably be in the form of a tablet, which will dissolve in the feed of the animal, or else a tablet to be crunched by the latter.

According to yet another aspect of the invention, it is envisaged to use a composition according to the invention containing an active principle based on calcium and/or magnesium pidolate, for stimulating, around the time of parturition, a general mobilization of calcium ions in a nonhuman female mammal, preferably a farm animal. More preferably, the nonhuman female farm mammal is a cow and the composition is in the form of a bolus. According to a preferred variant of this use, the nonhuman female farm mammal is a sow and the composition is in tablet form.

According to yet another aspect of the present invention, it is envisaged to use a composition according to the invention, containing an active principle based on calcium and/or magnesium pidolate, for increasing the weight gain of a piglet suckling on the mother. According to yet another aspect, the invention relates to the use of a composition according to the invention, containing an active principle based on calcium and/or magnesium pidolate, for speeding up the parturition time of the animal, and preferably the sow.

According to two other aspects of the present invention, it is envisaged:
to use calcium and/or magnesium pidolate to maintain the blood calcium level peri-partum in the cow above 85 mg/l by supplying at least the equivalent of 3.78 g, preferably at least the equivalent of 7.56 g, of calcium in the form of pidolate salt;
to use calcium and/or magnesium pidolate to increase the blood phosphorus level peri-partum in the cow above 55 mg/l by supplying at least the equivalent of 30.24 g of calcium pidolate and/or 7.5 g of magnesium pidolate.

Thus, as can be seen from the foregoing, the applicant has developed several uses of calcium and/or magnesium pidolate in the area of animal feeding and animal health. It was found, when the two pidolates were used together, that it was preferable to use a ratio 4:1 of calcium pidolate relative to magnesium pidolate.

In order to support the applicant's position, it seems important to describe, in biochemical terms, the mechanisms regulating the blood levels of the macroelements such as Ca, P, Mg, called homeostasis. The vital functions of the organism also depend on the homeostasis of these elements. When the latter is disturbed, pathological events occur.

For example, 99% of the organic Ca is made up of the skeleton in the form of complex salts (hydroxyapatite), in ionized form, in a close relationship with P; Ca has multiple functions:
transmission of the nerve impulse
involvement in muscle contraction (smooth or striated)
regulation of the permeability of the cell membranes
regulation of the mechanisms of transduction of hormonal messages
participation in blood coagulation
enzyme activation
control of the use of ATP (close relation with the 3 phosphate groups of the structure of ATP).

The regulation of blood calcium, without causing injury to the cow, notably with regard to osteoporosis, is dependent on several factors: parathormone (PTH), calcitriol (1,25-dihydroxyvitamin D) and secondarily thyroid calcitonin. Owing to the sensitive transmembrane receptors, when the blood calcium level decreases, secretion of PTH and synthesis of calcitriol increase significantly. PTH permits bone resorption of Ca and tubular reabsorption of Ca by the kidney. It has an effect on calcitriol synthesis. Calcitriol, produced by the kidney, stimulates intestinal absorption (duodenum-jejunum) of dietary Ca, and regulates bone resorption of Ca. Hypocalcemia and milk fever, or parturient paresis, occur when the Ca of osseous origin cannot be displaced and the diet does not compensate the loss of milk Ca. When the blood calcium level increases, secretion of PTH decreases, there is a decrease in bone resorption and tubular reabsorption, and thus the synthesis of calcitriol decreases, owing to its reduced intestinal absorption. Then thyroid calcitonin starts to be produced.

There are several factors affecting regulation by PTH:
hypomagnesemia hinders the response of the parathyroid. The Mg status decreases when absorption of K increases, for example on turning out to graze, which leads to hypocalcemia;
diets too high in Ca (alfalfa, excess of Ca-rich CMV), at the end of gestation, inhibit secretion of PTH. In contrast, diets low in Ca, for example less than 20 g of daily supply of Ca, stimulate secretion of PTH, permitting good osteoclasis and production of calcitriol, which improves the absorption of dietary Ca by the enterocytes;
metabolic alkalosis, which is defined by a urinary pH above 7.8, at positive BACA, by excess of K and deficiency of chlorides, predisposes to hypocalcemia and to parturient paresis;
controlled ruminal acidification and negative Baca (0.15 Ca+at 0 15 Mg=Na+K)−(Cl+at 0.25 S+0.5 P)=−200 mEQ/kg. Sulfates are less acidifying than chlorides (of Mg, of ammonium), permitting effective prevention of parturient paresis;
hepatic insufficiency (steatosis) decreases the synthesis of 25 hydroxyvitamin D which will be deficient at the renal level for production of 1,25 dihydroxyvitamin D or calcitriol;
corticoids are aggravating factors of hypocalcemia, notably in the case of muscular disorders or of inflammation of the nerve trunks.

There are also factors affecting the absorption of dietary Ca
with increase in age of the animal, the number of PTH receptors decreases, which is reflected in diminished maintenance of homeostasis in the animal;
estrogens, which increase at the moment of parturition, have an inhibitory action on the homeostatic response;
calcitonin secreted by the thyroid decreases osteoclasis and increases urinary excretion of Ca;
if acidosis is excessive, as the blood pH decreases, osteoclasis will be increased for a better acid-base balance of the blood, by the Ca ion.

There are also factors affecting renal production of calcitriol, as the latter is practically similar between a healthy animal and a cow with vitular neurosis, where the level is slightly raised:
a BACA+ reduces the synthesis of calcitriol, as alkaloses reduce its production and the sensitivity of the renal tissue to PTH;
high blood concentrations of P inhibit its activity and increase hypocalcemia.

It is known that magnesium participates in the metabolism of nucleic acids, in the organization of chromatin, in protein synthesis and in energy production at the level of the cytoplasm and mitochondria. It is a cofactor that is essential for the activity of many enzymes present in the cell nucleus, the mitochondria, the endoplasmic reticulum and the cytoplasm. It is also involved in the direct or indirect regulation of more than 300 enzymes (ATPases, protein kinase C etc.). It participates in stabilization of the structure of the proteins, nucleic acids and cell membranes. It controls the utilization of ATP, which serves as phosphate substrate or donor (ATP provided with 3 negatively charged phosphate groups is stabilized by the Mg ion). As a divalent cation, it is indispensable to the electrochemical stability of many charged molecules of the cell and to membrane cohesion. The osseous reserves of Mg are large, about 70% of the body's Mg, but short-term mobilizations are slight. If dietary supply of Mg is insufficient, if ruminal absorption of Mg is hampered by antagonists, the cow's blood status will be low. Below 18 mg/l, hypomagnesemia will give rise to hypocalcemia. Hypomagnesemia also interferes with the capacity of the Ca target cells to stimulate additional production of PTH, which causes a decrease in PTH. In hypocalcemia, this PTH nevertheless increases the reabsorption of Mg by the kidney, as renal excretion is slowed down.

To summarize, PTH causes hypercalcemia and hypophosphatemia, calcitriol causes hypercalcemia and hyperphosphatemia, calcitonin causes hypocalcemia and hypophosphatemia (inhibition of bone resorption) and phosphatonin causes hypophosphatemia.

To control the blood calcium level, blood phosphorus level and blood magnesium level during peripartum, it is indispensable:
to prevent any alkalosis, any hepatic steatosis or renal impairment toward the end of gestation;
to respect the mechanisms of production or of synthesis of parathormone and calcitriol, in the dairy cow at the end of gestation and more, by optimizing the supplies, neither too much nor too little, of Ca, P and Mg.

As mentioned above, the compositions according to the invention can integrate calcium and/or magnesium pidolate as active principles, which can then be used in the uses according to the invention. The calcium and/or magnesium pidolates used according to the invention have a maximum digestive bioavailability, making it possible to supply 13.5% of Ca and 8.5% of Mg, for example via 2 boli of 75 g, which can be metabolized directly by the enterocytes, and to capture, or chelate, the Ca, Mg and P present in the alimentary canal, via the protein complex of the pidolates. They also permit mobilization of bone calcium via carboxyglutamic acid and regulation of calcemia and phosphatemia, around parturition (peri-partum), as was demonstrated by zootechnical tests conducted by the applicant and described below. Moreover, the compositions of the invention containing calcium and/or magnesium pidolate in the form of a bolus permit controlled release, with a single dose, which avoids the burst effect and the drop in the corresponding blood levels. Secondly, they cause a significant increase of the growth hormone IGF1 (insulin growth factor) precursor, involved in the formation of bone and muscle cells.

### Tests in Cows

Tests with administration of a composition according to the invention in the form of a bolus containing calcium and magnesium L-pidolate in a 4:1 mixture were conducted on cows around calving. These tests were conducted in parallel with tests comparing the efficacy of two products already commercialized and available on the market, namely Bovicalc and Calform-phosphorus.

The tests were conducted on 5 groups of 6 multiparous dairy cows, at peripartum, high-yielders, which have calved at least once, from the same farm and not receiving any product based on Ca or Mg other than those stated below. One group out of the 5 test groups did not receive any product and is regarded as the control group.

Each group is identified with a letter, namely N, O, P, Q and R. The operations started about 2 to 3 hours before calving. The moment of calving was noted, indicated as H0, and blood tests were carried out around parturition and up to 25 hours afterward. These periods of blood sampling are indicated by reference to calving, therefore reference H, and are therefore:
H-4 to 5: 4 to 5 hours before calving;
H0: moment of calving;
H+3 to 4: 3 to 4 hours after calving;
H+12 to 13: 12 to 13 hours after calving;
H+22 to 25: 22 to 25 hours after calving.

The various products were administered as follows:
group N: two boli according to the invention in a single dose at the first signs of calving, immediately after collecting the blood samples, the reference of the bolus according to the invention being 7040-2-13 (13003), or 7.56 g of Ca, and 1.19 g of Mg;
group O: three boli according to the invention in a single dose at the first signs of calving, immediately after collecting the blood samples, the reference of the bolus according to the invention being 7040-2-13 (13003), or 11.34 g of Ca, and 1.78 g of Mg;
group P: control group, no product administered;
group Q: one Bovicalc bolus, at the first signs of calving, immediately after collecting the blood samples, then a second Bovicalc bolus 4 to 5 hours after calving, then finally a third Bovicalc bolus 12 hours after calving, immediately after collecting the blood samples, according to the supplier's recommendations;
group R: one 350 ml bottle of Calform, at the first signs of calving, and after collecting the blood samples, a second 350 ml bottle of Calform 4 to 5 hours after calving, then a third 350 ml bottle of Calform 12 hours after calving, immediately after collecting the blood samples, following the supplier's recommendations.

The three administrations of Bovicalc supplied a total of 129 g of Ca contained in the three boli, and the three bottles of Calform-phosphorus supplied 156 g of Ca. In terms of supply of Mg, the 3 bottles of Calform-phosphorus supplied 2.4 g. The boli according to the invention did not supply phosphorus, compared to the 135 g of P of the 3 bottles of Calform-phosphorus.

The bolus according to the invention, bearing the reference 7040-2-13 (13003), had the following composition:

TABLE 1
Bolus 7040-2-13 (13003)

| Name | Value |
|---|---|
| Reference | 7040-2-13 (13003) |
| Active principle - Ca/Mg pidolate | 50.00% |
| (4:1 mixture) | |
| Accelerating agent - Arbo C12 | 12.00% |
| lignosulfonate | |
| Delaying agent - Hydrogenated soya | 8.00% |
| oil | |
| Ballast - iron particles | 29.00% |
| Excipients | 1.00% |
| TOTAL | 100.00% |
| Solubility Index | 27.32 |
| Disintegration time | 33 hours |

TABLE 2
Calcemia Results Group N

| | Part. | H − 4 | | H + 3 | H + 12 | H + 22 |
|---|---|---|---|---|---|---|
| Identity | Date | to H − 1 | H 0 | to 4 | to 13 | to 25 |
| 8595 | 20-May | 85 | 85 | 86 | 83 | 83 |
| 8662 | 28-Aug | 88 | 79 | 80 | 79 | 76 |
| 5282 | 02-Sep | 82 | 82 | 83 | 84 | 74 |
| 9467 | 06-Oct | 84 | 76 | 80 | 75 | 69 |
| 8855 | 16-Oct | 83 | 82 | 89 | 75 | 80 |
| 9576 | 07-Jan | 82 | 81 | 78 | 84 | 85 |
| Group N | | 84.0 | 80.8 | 82.7 | 80.0 | 77.8 |

TABLE 3
Calcemia Results Group O

| | Part. | H − 4 | | H + 3 | H + 12 | H + 22 |
|---|---|---|---|---|---|---|
| Identity | Date | to H − 1 | H 0 | to 4 | to 13 | to 25 |
| 8664 | 19-May | | 72 | 66 | 69 | 62 |
| 9559 | 12-Sep | 99 | 90.0 | 93.0 | 101.0 | 88.0 |
| 9462 | 03-Oct | 98 | 92.0 | 95.0 | 94.0 | 90.0 |
| 7820 | 11-Oct | 92 | 85.0 | 93.0 | 89.0 | 90.0 |
| 9454 | 16-Oct | 83 | 84.0 | 82.0 | 81.0 | 78.0 |
| 8849 J | 17-Oct | 92 | 93.0 | 93.0 | 89.0 | 79.0 |
| Group O | | 92.8 | 86.0 | 87.0 | 87.2 | 81.2 |

TABLE 4
Calcemia Results Group P

| | Part. | H − 4 | | H + 3 | H + 12 | H + 22 |
|---|---|---|---|---|---|---|
| Identity | Date | to H − 1 | H 0 | to 4 | to 13 | to 25 |
| 9320 | 26-Aug | 91 | 90 | 85 | 86 | 81 |
| 9413 | 28-Oct | 81 | 76 | 74 | 73 | 71 |
| 8476 | 15-Nov | 84 | 85 | 82 | 86 | 80 |
| 8489 | 21-Nov | 84 | 81 | 74 | 74 | 85 |
| 9552 | 22-Nov | 84 | 90 | 88 | 84 | 77 |
| 9315 | 08-Feb | 57 | 53 | 46 | 41 | 56 |
| Group P | | 80.2 | 79.2 | 74.8 | 74.0 | 75.0 |

TABLE 5
Calcemia Results Group Q

| | Part. | H − 4 | | H + 3 | H + 12 | H + 22 |
|---|---|---|---|---|---|---|
| Identity | Date | to H − 1 | H 0 | to 4 | to 13 | to 25 |
| 8849 S | 31-Aug | 87 | 87 | 87 | 91 | 90 |
| 8846 | 29-Oct | 93 | 96 | 85 | 93 | 86 |
| 9439 | 05-Nov | 64 | 65 | 72 | 75 | 77 |
| 9538 | 20-Feb | 89 | 93 | 96 | 86 | 91 |
| 8670 | 25/04 | 85 | 86 | 86 | 90 | 91 |
| 9406 | 27/04 | 75 | 83 | 85 | 88 | 92 |
| Group Q | | 82.2 | 85 | 85.2 | 87.2 | 87.8 |

TABLE 6
Calcemia Results Group R

| | Part. | H − 4 | | H + 3 | H + 12 | H + 22 |
|---|---|---|---|---|---|---|
| Identity | Date | to H − 1 | H 0 | to 4 | to 13 | to 25 |
| 9556 | 10-Oct | 92 | 93 | 106 | 93 | 84 |
| 8851 | 10-Nov | 83 | 81 | 74 | 81 | 73 |
| 8590 | 23-Nov | 86 | 90 | 88 | 95 | 87 |
| 9433 | 25-Dec | 80 | 83 | 81 | 90 | 108 |
| 9480 | 25-Jan | 84 | 85 | 86 | 82 | 83 |
| 8531 | 26-Jan | 86 | 87 | 92 | 93 | 90 |
| Group R | | 85.2 | 86.5 | 87.8 | 89.0 | 87.5 |

### Calcemia Comparison, Group N Versus Group P, Group O, Group R

The blood calcium levels for group N are slightly higher than those for the control group P, but although being in a zone with risk of hypocalcemia, i.e. with a blood calcium level below 85 mg/l, the blood Ca values were maintained at a good level and there was no morbid manifestation, in contrast to the control group, in which one cow out of six had milk fever. The blood calcium levels for groups Q and R are slightly higher (7 to 11%) than those for group N, in which the subjects only had a single administration of a bolus according to the invention. It is astonishing, bearing in mind the massive supply of Ca from Bovicalc (17 times more) and from Calform-phosphorus (20 times more), that the blood Ca levels barely exceed 92 mg/l for these two products.

### Calcemia Comparison, Groups N and O Versus Groups P, Q, R

The blood calcium levels for group O are at higher levels than those for group N and group P, as the blood levels of the cows taken at H-4 to H-1 are above 90 mg/l, thus showing a lower risk for the subjects in this group, but the values show that the blood profile is roughly parallel to that of group N. It is therefore certain that the bolus according to the invention based on Ca and Mg pidolates, disintegration of which takes place over about 30 hours, has a direct influence on homeostasis of Ca and P.

### Comparison of Low Blood Calcium Levels in Each Group:

TABLE 7
Low blood calcium levels - Group N

| | Part. | H − 4 | | H + 3 | H + 12 | H + 22 |
|---|---|---|---|---|---|---|
| Identity | Date | to H − 1 | H 0 | to 4 | to 13 | to 25 |
| 5282 | 02-Sep | 82 | 82 | 83 | 84 | 74 |
| 9467 | 06-Oct | 84 | 76 | 80 | 75 | 69 |
| 8855 | 16-Oct | 83 | 82 | 89 | 75 | 80 |
| 9576 | 07-Jan | 82 | 81 | 78 | 84 | 85 |
| Group N | | 82.8 | 80.3 | 82.5 | 79.5 | 77.0 |

TABLE 8
Low blood calcium levels - Group O

| | Part. | H − 4 | | H + 3 | H + 12 | H + 22 |
|---|---|---|---|---|---|---|
| Identity | Date | to H − 1 | H 0 | to 4 | to 13 | to 25 |
| 9454 | 16-Oct | 83 | 84.0 | 82.0 | 81.0 | 78.0 |
| Group 0 | | 83.0 | 78.0 | 74.0 | 75.0 | 70.0 |

TABLE 9
Low blood calcium levels - Group P

| | Part. | H − 4 | | H + 3 | H + 12 | H + 22 |
|---|---|---|---|---|---|---|
| Identity | Date | to H − 1 | H 0 | to 4 | to 13 | to 25 |
| 9413 | 28-Oct | 81 | 76 | 74 | 73 | 71 |
| 8476 | 15-Nov | 84 | 85 | 82 | 86 | 80 |
| 8489 | 21-Nov | 84 | 81 | 74 | 74 | 85 |
| 9552 | 22-Nov | 84 | 90 | 88 | 84 | 77 |
| 9315 | 08-Feb | 57 | 53 | 46 | 41 | 56 |
| Group P | | 78.0 | 77.0 | 72.8 | 71.6 | 73.8 |

TABLE 10
Low blood calcium levels - Group Q

| | Part. | H − 4 | | H + 3 | H + 12 | H + 22 |
|---|---|---|---|---|---|---|
| Identity | Date | to H − 1 | H 0 | to 4 | to 13 | to 25 |
| 9439 | 05-Nov | 64 | 65 | 72 | 75 | 77 |
| 9406 | 27/04 | 75 | 83 | 85 | 87.7 | 92 |
| Group Q | | 69.5 | 74 | 78.5 | 81.5 | 84.5 |

TABLE 11
Low blood calcium levels - Group R

| | Part. | H − 4 | | H + 3 | H + 12 | H + 22 |
|---|---|---|---|---|---|---|
| Identity | Date | to H − 1 | H 0 | to 4 | to 13 | to 25 |
| 8851 | 10-Nov | 83 | 81 | 74 | 81 | 73 |
| 9433 | 25-Dec | 80 | 83 | 81 | 90 | 108 |
| 9480 | 25-Jan | 84 | 85 | 86 | 82 | 83 |
| Group R | | 82.3 | 83.0 | 80.3 | 84.3 | 88.0 |

The 4 blood calcium levels for group N are 5 to 12% higher relative to the 5 for group P, in the 4 to 12 h following calving. Those for groups Q and R show a good increase especially starting from the twelfth hour, the blood calcium levels of the cows in group Q being very low before calving. Moreover, those for cow No. 9315 in group P are representative of the course of milk fever, which was detected and treated about twelve hours after calving.

### Comparison of Blood Phosphorus Levels in Groups N and O Versus Groups P, Q, R:

TABLE 12
Blood phosphorus level - Group N

| | Part. | H − 4 | | H + 3 | H + 12 | H + 22 |
|---|---|---|---|---|---|---|
| Identity | Date | to H − 1 | H 0 | to 4 | to 13 | to 25 |
| 8595 | 20-May | 41.9 | 38.6 | 57.2 | 55.8 | 48.8 |
| 8662 | 28-Aug | 50.9 | 41.3 | 48.9 | 57.7 | 58.4 |
| 5282 | 02-Sep | 82.1 | 69.3 | 82.2 | 76.8 | 72.4 |
| 9467 | 06-Oct | 43.3 | 38.5 | 58.8 | 52.5 | 49.3 |
| 8855 | 16-Oct | 42.3 | 42.2 | 51.0 | 57.4 | 51.4 |
| 9576 | 07-Jan | 83.4 | 78.9 | 81.2 | 61.9 | 53.5 |
| Group N | | 57.3 | 51.5 | 63.2 | 60.3 | 55.6 |

TABLE 13
Blood phosphorus level - Group O

| | Part. | H − 4 | | H + 3 | H + 12 | H + 22 |
|---|---|---|---|---|---|---|
| Identity | Date | to H − 1 | H 0 | to 4 | to 13 | to 25 |
| 8664 | 19-May | 67 | | 53.5 | 69.9 | 56.6 |
| 9559 | 12-Sep | 69.9 | 75.4 | 82.6 | 106.4 | 82.8 |
| 9462 | 03-Oct | 65.1 | 60.8 | 69.4 | 97.0 | 53.6 |
| 7820 | 11-Oct | 64.6 | 47.6 | 69.7 | 61.7 | 56.1 |
| 9454 | 16-Oct | 48.5 | 40.2 | 47.3 | 64.2 | 46.1 |
| 8849 J | 17-Oct | 71.4 | 53.7 | 84.5 | 76.1 | 54.7 |
| Group O | | 64.4 | 55.6 | 67.8 | 79.2 | 58.3 |

TABLE 14
Blood phosphorus level - Group P

| | Part. | H − 4 | | H + 3 | H + 12 | H + 22 |
|---|---|---|---|---|---|---|
| Identity | Date | to H − 1 | H 0 | to 4 | to 13 | to 25 |
| 9320 | 26-Aug | 41.7 | 43.5 | 44.1 | 43.3 | 45.9 |
| 9413 | 28-Oct | 51.2 | 54.3 | 43.8 | 47.6 | 50.6 |
| 8476 | 15-Nov | 54.9 | 58.1 | 62.0 | 66.1 | 67.8 |
| 8489 | 21-Nov | 58.2 | 55.8 | 61.3 | 79.3 | 68.2 |
| 9552 | 22-Nov | 46.4 | 54.5 | 68.2 | 50.6 | 45.9 |
| 9315 | 08-Feb | 37.7 | 51.3 | 29.3 | 17.6 | 33.5 |
| Group P | | 48.4 | 52.9 | 51.4 | 50.7 | 52.0 |

TABLE 15
Blood phosphorus level - Group Q

| | Part. | H − 4 | | H + 3 | H + 12 | H + 22 |
|---|---|---|---|---|---|---|
| Identity | Date | to H − 1 | H 0 | to 4 | to 13 | to 25 |
| 8849 S | 31-Aug | 53.5 | 50.5 | 52.9 | 39.1 | 42.0 |
| 8846 | 29-Oct | 67.2 | 57.4 | 50.3 | 59.1 | 44.6 |
| 9439 | 05-Nov | 22.5 | 15.3 | 17.9 | 24.1 | 36.3 |
| 9538 | 20-Feb | 36.9 | 43.5 | 42.5 | 47.6 | 43.8 |
| 8670 | 25/04 | 33.45 | 53.86 | 58.27 | 57.37 | 39.22 |
| 9406 | 27/04 | 50.6 | 63.4 | 79.58 | 89.97 | 92.57 |
| Group Q | | 44 | 47.3 | 50.1 | 52.94 | 49.7 |

TABLE 16
Blood phosphorus level - Group R

| | Part. | H − 4 | | H + 3 | H + 12 | H + 22 |
|---|---|---|---|---|---|---|
| Identity | Date | to H − 1 | H 0 | to 4 | to 13 | to 25 |
| 9556 | 10-Oct | 74.1 | 71.7 | 92.9 | 78.6 | 51.4 |
| 8851 | 10-Nov | 39.5 | 43.4 | 50.8 | 45.1 | 41.0 |
| 8590 | 23-Nov | 49.9 | 52.3 | 45.3 | 61.8 | 52.3 |
| 9433 | 25-Dec | 77.6 | 71.9 | 77.3 | 70.7 | 71.7 |
| 8531 | 25-Jan | 45.1 | 58.5 | 66.4 | 64.6 | 56.6 |
| 9480 | 26-Jan | 25.2 | 25.3 | 37.7 | 38.6 | 28.2 |
| Group R | | 51.9 | 53.8 | 61.7 | 59.9 | 50.2 |

The blood phosphorus levels of group N, which did not receive any phosphorus supply, are comparable, or even slightly higher than those of group R, which received 3 administrations of 135 g of phosphorus. They are well above the control group P and especially group Q, which also did not receive any phosphorus supply. The blood phosphorus levels of group N and those of Group O are comparable at different levels. The dose effect is clearly seen in group O. It is therefore evident that the pidolates mobilize endogenous organic phosphorus or induce its production.

Except for group O, which only has one animal at risk, it is interesting to compare the blood phosphorus levels at risk, i.e. below 55 mg/l, or at high risk, i.e. below 40 mg/l, in each group. The values for group N, after a drop in blood levels at the time of calving, i.e. 1 to 2 hours after a single administration of the two boli, recover markedly and exceed that of group R with supply of 135 g of phosphorus in three administrations. Group O with a single subject was not taken into account, but is nevertheless very representative. The mobilization of endogenous organic phosphorus by the pidolates is confirmed.

Results for the Behavior of the Cows after Calving:

The cows were investigated for their physical behavior at the time of, and after, calving. The results of this study were expressed as a percentage relative to 100% representing the behavior of a cow in ideal physical condition. The values for groups N and O were clearly positive relative to the other three groups. In fact, the physical behaviors of the cows in groups N and O were 75% and 72% relative to an ideal physical condition, compared to 38.9%, for group P, to 33.3% for group Q, the latter displaying mastitis and metritis after calving, and to 55.6% for group R.

These good results are to be attributed to the Ca and Mg pidolates, which have a very good effect on homeostasis of Ca, P and Mg. As shown by the excellent biochemical results for blood phosphorus levels of groups N and O, the metabolic phosphorus exacerbated by the pidolates stimulates mastication, rumination and digestion by multiplication of the digestive flora and moreover 5-oxo-1-proline has, via glutathione, a clear effect on the oxidative stress inherent in calving.

### Comparison of Milk Production by the INRA Method:

At the instigation of the French milk inspection agency, a method of calculation based on lactation on the 4th, 5th, and 6th day after calving was developed and proposed by INRA (Institut National de la Recherche Agronomique—National Agricultural Research Institute), to identify the “lactation peak” and better define future milk production.

TABLE 17
Milk production, days 4, 5, 6 post-partum

| | | Group | Group | Group | Group | Group |
|---|---|---|---|---|---|---|
| | | N | O | P | Q | R |
| Prod D 4 | 12 | 30.4 | 29.4 | 26.1 | 23 | 29.1 |
| Prod D 5 | 15 | 31.1 | 29.6 | 25.5 | 24.8 | 29.4 |
| Prod D 6 | 16 | 33.6 | 32.9 | 28.7 | 25.7 | 33 |
| Mean value | 14.33 | 31.70 | 30.63 | 26.77 | 24.50 | 30.50 |
| Max. | 25.04 | 39.63 | 38.73 | 35.48 | 33.58 | 38.62 |
| potential | | | | | | |
| milk prod | | | | | | |
| Potential | 11.72 | 41.49 | 40.55 | 37.15 | 35.16 | 40.44 |
| milk prod | | | | | | |
| Actual | 5609 | 8877 | 8676 | 7948 | 7522 | 8651 |
| Lactation | | | | | | |
| Milk prod | | 6827 | 7082 | 7326 | 7449 | 7895 |
| N-1 | | | | | | |
| TOTAL | | 8877 | 8676 | 8074 | 7999 | 8651 |

Thus, it was found on the one hand that the mean value (of the 4th, 5th, and 6th days) of each group at NO is almost equivalent for groups N (31.7), O and R, compared to group P (26.7), and to group Q (24.5), or a daily difference of 5 to 71 of extra milk, and that on the other hand the forecasts for lactation are favorable for group N, with +226 liters relative to group R, and 800-870 liters relative to group P and Q. A comparison of the yields of each group during the first 8 days found the same phenomenon. It follows from this that the use of the pidolates according to the invention can stimulate milk production in larger quantities very soon after calving, on days 4 and 5 in particular, which has an appreciable effect on the overall yield and profitability of the animal, as well as the quality of the milk produced.

The actual production in the previous year (year N−1) and the forecasts for the cows in the test were compared, and the results are presented below:

TABLE 18
Comparison of Production N-1

| Group | N-1 in kg | Forecast | Difference | % |
|---|---|---|---|---|
| N | 6827 | 8877 | 2050 | 30 |
| O | 7082 | 8676 | 1594 | 23 |
| P | 7326 | 7948 | 622 | 8 |
| Q | 7449 | 7522 | 73 | 1 |
| R | 7895 | 8651 | 756 | 10 |

As can be seen, the administration of calcium and/or magnesium pidolates according to the present invention in the form of two or three boli makes it possible to significantly increase the amount of milk, relative to the commercially available products, which only supply calcium.

### Conclusions:

The pidolates of the invention used as supplement give equal performance, or even better performance than the existing products, notably Calform and Bovicalc, both in supply of calcium and control of blood calcium levels, and for management of blood phosphorus levels, notably during calving. This is important as it is known that milk fever or parturient paresis, as well as hypophosphoremia, and “downer cow” syndrome, are complications of hypocalcemia in 55% of the cases found. The 3 zootechnical comparisons demonstrate a clear superiority of groups N and O relative to the control group P and to group Q, but also better results compared to group R. Without wishing to be bound to a theory, the applicant thinks that this superiority is to be attributed not only to the induction, or mobilization, of the production of endogenous calcium, but also to the mobilization of endogenous organic phosphorus. It also cannot be ruled out that by the same pathway, bone resorption of calcium by hydroxyproline, and the antistress action of 5-oxo-1-proline permit rapid recovery of the animal. Moreover, in addition to their undeniable effects on the milk production behavior of the cows, the pidolates according to the invention, when administered in the form of boli, are done so easily, without danger, and just once, at the moment when the animal is most available, in contrast to the three or four administrations recommended or usual for the commercial products.

### Tests on Sows/Piglets

Tests with a composition according to the invention were also conducted on sows at the time of becoming mothers, about seven days before farrowing of the piglets, which are then suckled by the sow. The composition was administered in the form of tablets, which were either dissolved in the animals' feed, or were crunched directly by the animals.

### Sow Test 1001

Test 1001 consisted of recording the productivity and behavior at the time of farrowing of a group of 24 treated sows, designated group A, relative to that of 23 control sows, designated group B. The piglets in group A showed an average weight per piglet at weaning more than 5% greater than that of the untreated group.

### Sow Test 1003

Two groups of piglets were selected to constitute 2 test populations, with 2 groups per population. The first test population comprised 12 sows, divided into 2 separate groups of 6 sows, each sow being identified. The first group, called group 1A, consisted of 6 sows. Group 1A received the tablets according to the invention by the oral route for 14 days, added to the drinking water or on the feed pellets, at a rate of 1 tablet per day beginning 7 days before farrowing, or when the animals became mothers, and ending 7 days after farrowing. The identification and individual weighing of the piglets were carried out on the first day and at weaning, at about 28 days. The second group, called group 1B, also consisted of 6 sows. Group 1B received a placebo tablet, administered by the oral route for 14 days, added to the drinking water or on the feed pellets, at a rate of 1 tablet per day beginning 7 days before farrowing and ending 7 days after farrowing. Identification and individual weighing of the piglets were carried out on the first day and at weaning at 28 days.

For the second population, the first group, called group 2A, consisted of 6 sows. This group 2A received the tablets according to the invention administered by the oral route for 14 days, added to the drinking water or on the feed pellets, at a rate of 1 tablet per day beginning 7 days before farrowing and ending 7 days after farrowing. The piglets were not identified, but instead each group was weighed on the first day and at weaning at 28 days. The second group, called group 2B, consisted of 6 sows. This group 2B received a placebo tablet, administered by the oral route for 14 days, added to the drinking water or on the feed pellets, at a rate of 1 tablet per day beginning 7 days before farrowing and ending 7 days after farrowing. The piglets were not identified, but each group of piglets per sow was weighed on the first day and at weaning at 28 days.

The sows were examined and assessed during farrowing according to the following criteria:
Date of farrowing
Duration of farrowing
Ease of recovery
Record of treatments carried out or obstetric interventions
Return of appetite
State of the mammary gland
Checking of rectal temperature at the end of farrowing and 12 hours later
Treatment of the sows with temperature above 39.3° C.
Cannibalism

Next, the following assessments were carried out:
During the last farrowing
   Piglets born, total
   Piglets stillborn
   Piglets weaned
At birth
   Litter order
   Number born
   Number of stillbirths
   Identification of each piglet of GROUP No. 1
   Weighing of each piglet of group No. 1
   Weighing of the litter of Group No. 2
   Uniformity
at 3 days and 7 days: morbid manifestations (diarrhea, omphalitis, arthritis)
at 28 days:
   date of weaning
   number of piglets present
   weighing of each piglet of group No. 1
   weighing of the litter of group No. 2
   uniformity
at slaughter: if possible, the carcass weights of the individuals of group No. 1, A and B, identified on the first day, were checked

The composition according to the invention that was administered was as follows:

TABLE 19
Formulation VST 212

| Name | Value |
|---|---|
| Reference | VST 212 |
| Active principle - Ca pidolate (12.5% Ca) | 60.00% |
| Accelerating agent - Arbo C12 | 4.00% |
| Lignosulfonate | |
| Delaying agent - Hydrogenated soya oil | 4.50% |
| Compression agent - anhydrous dicalcium | 12.13% |
| phosphate | |
| Excipients | 19.37% |
| TOTAL | 100.00% |
| Solubility Index | 27.32 |

The results of this test are presented below:

Population 1, Group A (became mothers on 21/10)-72 piglets weaned

TABLE 20
Population 1 - Group A - Weight

| | Number of | | | | |
|---|---|---|---|---|---|
| | days | | Weight | | |
| Sow | before | Weight | at | Weight | |
| No. | farrowing | D1 | weaning | gain | GMQ |
| 7362 | 6 | 1.18 | 8.48 | 7.23 | 0.249 |
| 8600 | 8 | 1.2 | 7.66 | 6.59 | 0.244 |
| 9756 | 6 | 1.76 | 7.91 | 6.15 | 0.212 |
| 9841 | 8 | 1.98 | 9.29 | 7.33 | 0.272 |
| 8595 | 7 | 1.24 | 8.62 | 7.31 | 0.261 |
| 9942 | 4 | 1.34 | 7.68 | 6.31 | 0.204 |
| Mean | 6.5 | 1.45 | 8.67 | 6.82 | 0.240 |

Population 1, Group B (became mothers on 21/10)-66 piglets weaned

TABLE 21
Population 1 - Group B - Weight

| | Number of | | | | |
|---|---|---|---|---|---|
| | days | | Weight | | |
| Sow | before | Weight | at | Weight | |
| No. | farrowing | D1 | weaning | gain | GMQ |
| 9943 | | | | | |
| 7390 | 8 | 1.59 | 8.15 | 6.49 | 0.241 |
| 8690 | 10 | 1.41 | 6.03 | 4.59 | 0.184 |
| 9758 | 8 | 1.23 | 7.63 | 6.39 | 0.237 |
| 9837 | 8 | 1.34 | 7.33 | 5.97 | 0.221 |
| 9836 | 8 | 1.24 | 7.68 | 6.41 | 0.237 |
| Mean | 8.4 | 1.36 | 7.36 | 5.97 | 0.224 |

Comparison Group 1A and Group 1B

TABLE 22
Synoptic comparison - Population 1

| Total | Number of days | | | | |
|---|---|---|---|---|---|
| population: | before | Weight | Weight at | Weight | |
| 138 | farrowing | D1 | weaning | gain | GMQ |
| Diff Days | −1.900 | 0.087 | 0.912 | 0.852 | 0.016 |
| Difference % | −22.62% | 6.42% | 12.39% | 14.27% | 7.37% |

As can be seen, the sows in group 1A, which received a composition based on calcium and/or magnesium pidolate according to the invention, had a shorter farrowing time than those that only had the placebo. Moreover, the weight gain in the piglets suckled by the sows that received calcium and/or magnesium pidolate was significantly greater, of the order of 14% extra weight, compared to their homologs, for which the mother sows only received the placebo.

Population 2, Groups A and B (became mothers on 21/10)—144 piglets weaned in total

TABLE 23
Population 2 - Comparison Group A - Weight

| Number of | Number of days | | | | |
|---|---|---|---|---|---|
| piglets | before | Weight | Weight | Weight | |
| weaned | farrowing | D1 | at weaning | gain | GMQ |
| Group A: | 7.000 | 1.270 | 7.230 | 5.960 | 0.213 |
| 74 | | | | | |
| Group B: | 8.000 | 1.440 | 6.910 | 5.470 | 0.201 |
| 70 | | | | | |
| Diff Days | −0.830 | −0.173 | 0.315 | 0.489 | 0.012 |
| Diff % | −10.640 | −12.030 | 4.560 | 8.940 | 5.970 |

As can be seen, in group 2A, for which the sows received the calcium and/or magnesium pidolates according to the invention, farrowing took less time than for the placebo group. Moreover, the piglets in group A also showed a greater weight gain than their homologs in group 2B.

These results clearly show that the calcium and/or magnesium pidolates as used according to the invention have an appreciable effect on piglet growth, notably on higher carcass weight of the finished pig for one and the same fattening time, or on shorter fattening time for one and the same carcass weight. This also has a positive effect on the general health of the sow, since the shorter farrowing times are evidence of better recovery of the animal following the stress induced by the start of motherhood and farrowing. This recovery of health has important consequences for the farmer, both in terms of the cost of supervision of the animal, but also in terms of overall productivity of his farming operations.

Finally, during development of the compositions according to the present invention, the applicant also found it possible to measure the in vitro disintegration of the composition reproducibly and thus make it possible to predict, with a fair degree of certainty, the disintegration time in vivo, once the composition has been ingested by the animal. As far as the applicant knows, this is the first time that this has been achieved. In fact, it would appear that the prior art on this subject is limited to reproducing a synthetic salivary buffer proposed in an article published in 1948, by McDougall, E.I., with the title “Studies on ruminant saliva. The composition and output of sheep's saliva” published in Biochem. J., 43: 99-109. This article describes a set of components representing a synthetic “salivary” buffer, which is supposed to constitute the dissolution medium in the sheep, and which has a pH around 8. Paradoxically, the model that is proposed there and that has been used for years for dissolution tests and studies of active principles intended for ruminants in vitro is, basically, a salivary buffer. Since then, this buffer has been taken as a starting base by others, and modified, adjusting it to a pH closer to the pH of the rumen. Now, the applicant noticed that the buffer initially proposed by McDougall, and later modified by others by adding acetic acid, has a large pH shift with time, so that it no longer even performs its role of buffer. In fact, the main role of a buffer is to present roughly constant behavior over a relatively narrow and well defined pH range. For ruminants, the temperature and pH of the rumen are generally between about 38 degrees Celsius and about 41 degrees Celsius, for a pH between 5.8 and 6.4.

Thus, another object of the present invention is a method of measuring the disintegration in vitro of a composition according to the invention, comprising the steps consisting of:
preparing an aqueous buffer solution;
introducing a composition according to the invention into the buffer solution;
keeping the mixture of buffer and composition at constant temperature and stirring;
determining the disintegration of the composition at regular intervals until it has disintegrated completely.

Several additional and preferred steps can be added to the method described above. According to a first preferred alternative, the method according to the invention further comprises a step consisting of replacing the buffer solution with an amount of buffer solution that is equivalent in volume every hour after initial introduction of the composition into the buffer solution.

According to another preferred variant, the method further comprises a step consisting of replacing the buffer solution with an amount of buffer solution that is equivalent in volume every twelve hours after initial introduction of the composition into the buffer solution.

According to yet another preferred variant, the method further comprises a step consisting of replacing the buffer solution with an amount of buffer solution that is equivalent in volume every forty-eight hours after initial introduction of the composition into the buffer solution.

According to yet another preferred variant, the method further comprises a step consisting of replacing the buffer solution with an amount of buffer solution that is equivalent in volume every seventy-two hours after initial introduction of the composition into the buffer solution.

Whichever variants are used, it is also preferred that the method further comprises a step consisting of removing the solid residue of the composition on replacement of the buffer solution, of rubbing said solid residue to remove a surface film, and of re-introducing said residue into the buffer solution.

In fact, in the rumen, compositions of the bolus type are subjected to considerable friction effects, in addition to the constant movements of mixing and stirring.

Advantageously and preferably, the method is carried out while maintaining the temperature of the solution at 39 degrees Celsius.

Moreover, the stirring in the method is preferably carried out with a rotating magnetized bar, rotating at between 200 and 300 revolutions per minute.

According to other advantageous and preferred characteristics, disintegration is determined by weighing the composition before putting it in the buffer solution, then again during each replacement of buffer solution, the composition is in the form of a bolus, and the composition is put in a net before being introduced into the buffer solution. Advantageously, the composition is suspended in the buffer solution.

Preferably, the buffer solution used in the method according to the invention is composed of 0.2M Na₂HPO₄.2H₂O, 0.1M citric acid and NaCl 0.5 g/L. This buffer can be prepared in two ways as described below:
by direct mixing: to prepare 1 liter of buffer, weigh 22.25 g of disodium hydrogen phosphate dihydrate (Na₂HPO₄.2H₂O), 7.2 g of anhydrous citric acid, 0.5 g of anhydrous NaCl, and make up to 1 liter by adding distilled or deionized water. Then mix by stirring until completely dissolved and a translucent solution is obtained. The pH obtained is 5.80/5.85;
by adjustment: prepare a basic solution and an acid solution separately. The acid solution contains 0.1M citric acid and 0.5 g/liter NaCl in distilled or deionized water q.s. 1 liter, giving a solution with pH=2.30 approximately. The basic solution contains 0.2M disodium hydrogen phosphate dihydrate, 0.5 g/liter NaCl in distilled or deionized water q.s. 1 liter, giving a solution with pH=8.88 approximately.

After preparing the solutions, gradually pour, with stirring, the acid solution into the basic solution.

Monitor the variation of pH until the desired value is reached: 5.80/5.85, gradually lowering the pH by adding acid. About 1.2 liters of acid solution is required for 2 liters of basic solution.

For the purposes of the method of measurement, the composition is presented in the form of a bolus. Preferably, the bolus is enclosed and suspended in a net, for example a net for oranges. It is stirred in conical flasks with the buffer described above, for a volume of 3 liters. The system is stirred, for example with a magnetic bar, preferably with rotation on a stirring/heating plate, so as to provide stirring with a relatively small vortex. The stirring speed is preferably 200 to 300 revolutions/minute, and the measurement temperature is maintained at 39° C.+/−0.1 degree Celsius. Preferably, for the boli with short disintegration time, the dissolution/buffer solution is changed hourly with fresh buffer. According to another preferred variant of the invention for the medium-duration boli, the buffer solution is changed once every 24 hours with fresh buffer. According to another preferred variant of the invention, for the long-duration bolus, the buffer solution is changed every 48 hours with fresh solvent. The boli are generally weighed at T=0 for evaluating their dry weight. At each emptying: T+1, T+2, . . . T+n, etc., they are withdrawn from the solution, passed gently under the tap and/or rubbed gently between the fingers, to remove any surface film, then weighed “wet”, with the water of constitution that has penetrated into the matrix. The dissolution profiles are established and expressed as: % weight loss of the boli as a function of time/versus initial dry weight of the boli.

Moreover, in order to demonstrate the inability of the buffer proposed by McDougall to fulfill its role as buffer, tests were carried out with compositions according to the invention, in the form of a bolus and the buffer and the method according to the present invention. The details of these tests are given below:

Preparation of 10 L of McDougall buffer solution modified by adjusting the pH to 6.5 by adding acetic acid
1. Take 10 L of deionized water
2. Weigh the ingredients:
   NaHCO₃: 98 g
   Na₂HPO₄, 12H₂O: 93 g
   NaCl: 4.7 g
   KCl: 5.7 g
   CaCl₂, anhydrous: 0.4 g
   MgCl₂, anhydrous: 0.6 g
3. Dissolve the ingredients
4. Measure the pH: 8.24 at 20.8° C.
5. Adjust the pH to 6.50 with acetic acid
6. Homogenize
7. Heat the buffer to 39° C.: (temperature reached in 1 h): pH 6.75
8. Carry out disintegration of 2 boli whose composition complies with that according to the invention: references R12208 and R12210.

TABLE 24
Comparison of buffers

| | Disintegration | McDougall buffer | McDougall buffer | Vétalis buffer | Vétalis buffer |
|---|---|---|---|---|---|
| Bolus No. | time | measured pH | measured T° | measured pH | measured T° |
| R12208 | T0 | 6.75 | 39.0° C. | 5.84 | 39.0° C. |
| R12210 | (13 h 40) | 6.75 | 39.0° C. | 5.84 | 39.0° C. |
| R12208 | T + 30 min | 6.76 | 39.0° C. | 5.85 | 39.0° C. |
| R12210 | (14 h 10) | 6.78 | 39.0° C. | 5.85 | 39.0° C. |
| R12208 | T + 1 h | 6.79 | 39.0° C. | 5.86 | 39.0° C. |
| R12210 | (15 h 40) | 6.81 | 39.0° C. | 5.87 | 39.0° C. |
| R12208 | T + 2 h | 6.82 | 39.0° C. | 5.86 | 39.0° C. |
| R12210 | (16 h 40) | 6.82 | 39.0° C. | 5.85 | 39.0° C. |
| R12208 | T + 3 h | 6.90 | 39.1° C. | 5.87 | 39.1° C. |
| R12210 | (16 h 40) | 6.93 | 38.9° C. | 5.86 | 38.9° C. |
| R12208 | T + 4 h | 6.98 | 38.9° C. | 5.90 | 38.9° C. |
| R12210 | (17 h 40) | 6.98 | 38.9° C. | 5.89 | 39.1° C. |
| R12208 | T + 8 h | 7.34 | 38.9° C. | 5.97 | 39.0° C. |
| | (21 h 40) | (22 h 08) | | (22 h 12) | |
| R12210 | | 7.36 | 39.0° C. | 6.02 | 39.0° C. |
| | | (22 h 05) | | (21 h 50) | |
| R12208 | T + 1 day | 7.86 | 38.9° C. | 6.10 | 38.9° C. |
| R12210 | (13 h 40) | 8.17 | 39.1° C. | 6.03 | 38.9° C. |
| R12208 | T + 2 days | 8.32 | 39.1° C. | 6.23 | 39.1° C. |
| R12210 | (13 h 40) | 8.50 | 39.0° C. | 6.11 | 38.9° C. |
| R12208 | T + 3 days | 8.60 | 39.0° C. | 6.39 | 38.9° C. |
| | (13 h 40) | (14 h 18) | | (14 h 10) | |
| R12210 | | 8.72 | 39.1° C. | 6.20 | 38.9° C. |
| | | (14 h 30) | | (14 h 15) | |

As can be seen, the modified “buffer” solution, initially proposed by McDougall and adjusted by adding acetic acid, displays a large pH shift with time, changing from 6.75 at T0, to 8.72 at T+3D, which makes this buffer unsuitable as a representative buffer for studying the disintegration in vitro of compositions according to the invention, such as boli, intended for ruminants. Moreover, it should be noted that the McDougall buffer already shows a pH shift after raising to temperature, changing from pH 6.5 after adjustment with acetic acid to pH 6.75 after heating to 39 degrees Celsius. In contrast, the buffer developed by the applicant, whose pH at TO was 5.84, barely changes throughout disintegration of the test compositions, with pH measurement after three days of disintegration indicating a value of pH 6.20. Thus, the buffer developed by the applicant is entirely suitable for a protocol for measurement of disintegration in vitro, imitating that of ruminants for compositions according to the invention that are in the form of a bolus.

Examples of boli corresponding to the composition according to the invention were prepared and their details are given below. These examples were divided into categories of bolus, according to the disintegration time, i.e. according to the following classification:
bolus with disintegration between 1 hour and 30 days, called short-life bolus;
bolus with disintegration between 31 days and 90 days, called medium-life bolus;
bolus with disintegration between 91 days and 180 days, called long-life bolus;
bolus with disintegration longer than 180 days, called ultralong-life bolus.

All the compositions described can be obtained according to a general method of preparation, as detailed below:
1. Mixing of powders comprising: the active principle(s), the compression agent if any, optionally the disintegration accelerating agent, optionally the ballasting agent and the delaying agent, as well as an optional binder, in an apparatus of the mixer type, or in a mixer/granulator.
2. Optional granulation of the mixture thus obtained with water (or optionally aqueous alcohol of low titer), then drying in a stove set at about 50° C., or alternately drying in apparatus of the air fluidized bed type.
3. Optionally grinding and/or calibration of the dry or wet granules, to obtain particles passing through a sieve with size less than or equal to 4 mm, followed by optional drying, according to one of the two methods stated above.
4. Final mixing: starting from the particles obtained at the end of step 3, addition of lubricant as compression aid, disintegration delaying agent and optionally ballasting agent when the latter would be introduced during final mixing.
5. Optionally, in the case of tablets: compression of the mixture prepared in step 1; and for the boli: compression of the mixture starting from step 4, in a suitable die.

Other alternatives to the method described above can be envisaged:
Introduction of the ballasting agent and/or delaying agent, during step 1;
Introduction of the binder and/or the disintegration accelerating agent after dissolving and then spraying an aqueous (or aqueous-alcoholic) solution on the mixture to be granulated, at step 2;
Introduction of the delaying agent by the “hotmelt” technique (in the form of molten fat), during step 4, i.e. during final mixing.
   Short-life bolus (1 hour to 30 days)

Some examples of short-life formulations according to the invention, in the form of a bolus, are given below.

TABLE 25
Ingredients of Short-life bolus

| Product | Active principle | Accelerating agent | Delaying agent | Ballast |
|---|---|---|---|---|
| R1-01-02 | Copper glycinate | Arbo C12 (Na/NH₄) | Carnauba wax | Iron powder |
| R1-01-03 | Copper glycinate | Arbo C12 (Na/NH₄) | Hydrogenated palm oil | Iron powder |
| R1-01-06 | Copper glycinate | Arbo C12 (Na/NH₄) | Micronized stearin C16/C18 | Iron powder |
| R1-01-07 | Copper glycinate | Arbo C12 (Na/NH₄) | Paraffin | Iron powder |
| R1-01-04 | Copper glycinate | Arbo C12 (Na/NH₄) | Cetyl palmitate | Iron powder |
| R1-01-05 | Copper glycinate | Arbo C12 (Na/NH₄) | Glycerol trihydroxystearate | Iron powder |
| R1-07-03 | Copper glycinate | Arbo C12 (Na/NH₄) | Hydrogenated castor oil | Iron powder |
| R1-07-04 | Copper glycinate | Arbo C12 (Na/NH₄) | Stearic acid 92% | Iron powder |
| R1-07-01 | Copper glycinate | Arbo C12 (Na/NH₄) | Hydrogenated soybean oil | Iron powder |
| 7040-2-13 (13003) | Ca/Mg pidolate | Arbo C12 (Na/NH₄) | Hydrogenated soybean oil | Iron powder |
| 7040-2-13-6 (13003) | Ca/Mg pidolate | Arbo C12 (Na/NH₄) | Hydrogenated cottonseed oil | Iron powder |
| RI-47-02 bis(A) | Copper glycinate | Arbo C12 (Na/NH₄) | Hydrogenated soybean oil | Iron powder |
| R1-47-02-B (3.2) | Copper glycinate | Arbo C12 (Na/NH₄) | Hydrogenated soybean oil | Iron powder |
| R1-06-06 (b) | Copper glycinate | Arbo C12 (Na/NH₄) | Hydrogenated soybean oil | Iron powder |
| R1-06-01 | Copper glycinate | Ultrazine (Na) | Hydrogenated soybean oil | Iron powder |
| R1-06-02 | Copper glycinate | Ultrazine (Ca) | Hydrogenated soybean oil | Iron powder |
| R1-06-03 | Copper glycinate | Lignobond DD (Ca) | Hydrogenated soybean oil | Iron powder |
| R1-06-04 | Copper glycinate | Borresperse (Na) | Hydrogenated soybean oil | Iron powder |
| R1-06-05 | Copper glycinate | Borresperse AM320 | Hydrogenated soybean oil | Iron powder |
| | | (NH₄) | | |
| R1-07-05 | Copper glycinate | Arbo T11 N5 (NH₄) | Hydrogenated soybean oil | Iron powder |
| R1-07-06 | Copper glycinate | Arbo N18 Na | Hydrogenated soybean oil | Iron powder |
| R1-07-07 | Copper glycinate | Arbo K18 K | Hydrogenated soybean oil | Iron powder |
| R1-07-08 | Copper glycinate | Arbo C12 (Na/NH₄) | Hydrogenated soybean oil | Zinc powder |
| R1-02-01 | Copper glycinate | Arbo C12 (Na/NH₄) | Hydrogenated soybean oil | Iron powder |
| R1-09-10 | Anhydrous Mg | Arbo C12 (Na/NH₄) | Hydrogenated soybean oil | Iron powder |
| | sulfate | | | |
| R1-47-01 (bis) | Vitamin C | ArboC12 (Na/NH₄) | Hydrogenated soybean oil | Iron powder |
| R1-47-03 A (3.2) | Copper sulfate | Arbo C12 (Na/NH₄) | Hydrogenated soybean oil | Iron powder |
| R1-42-02 | Copper carbonate | Arbo C12 (Na/NH₄) | Hydrogenated soybean oil | Iron powder |
| R1-37-10 (A) | Cobalt carbonate | Arbo C12 (Na/NH₄) | Hydrogenated soybean oil | Iron powder |
| VST 224 | Pidolates of Ca | Arbo C12 | Hydrogenated colza oil | Iron powder |
| | 40%/and of Mg | | | |
| | 10% | | | |

The percentages of the various ingredients, their solubility index, and their disintegration times are given below:

TABLE 26
Amounts of Ingredients, Short-life bolus

| | | Accel. | Delaying | Comp. | Ballast | Excip. | Sol. | Time | |
|---|---|---|---|---|---|---|---|---|---|
| Product | P.A % | Agent % | agent % | Agent, % | % | % | Index | (h or d) | |
| R1-01-02 | 50 | 12 | 8 | 0 | 25.2 | 4.8 | 28.560 | 36 | h |
| R1-01-03 | 50 | 12 | 8 | 0 | 25.2 | 4.8 | 28.560 | 57 | h |
| R1-01-06 | 50 | 12 | 8 | 0 | 25.2 | 4.8 | 28.560 | 57 | h |
| R1-01-07 | 50 | 12 | 8 | 0 | 25.2 | 4.8 | 28.560 | 38 | h |
| R1-01-04 | 50 | 12 | 8 | 0 | 25.2 | 4.8 | 28.560 | 48 | h |
| R1-01-05 | 50 | 12 | 8 | 0 | 25.2 | 4.8 | 28.560 | 48 | h |
| R1-07-03 | 50 | 12 | 8 | 0 | 25.2 | 4.8 | 28.560 | 35 | h |
| R1-07-04 | 50 | 12 | 8 | 0 | 25.2 | 4.8 | 28.560 | 33 | h |
| R1-07-01 | 50 | 12 | 12 | 0 | 21.2 | 4.8 | 28.560 | 63.5 | h |
| 7040-2-13 (13003) | 50 | 12 | 8 | 0 | 29 | 1 | 27.315 | 33 | h |
| 7040-2-13-6 (13003) | 50 | 12 | 8 | 0 | 29 | 1 | 27.315 | 30 | h |
| R1-47-02 bis(A) | 50 | 12 | 5 | 0 | 28.2 | 4.8 | 28.560 | 72 | h |
| R1-47-02-B (3.2) | 50 | 12 | 8 | 0 | 25.2 | 4.8 | 28.560 | 93 | h |
| R1-06-06 (b) | 50 | 12 | 8 | 0 | 25.2 | 4.8 | 28.560 | 48 | h |
| R1-06-01 | 50 | 12 | 8 | 0 | 25.2 | 4.8 | 28.560 | 39 | h |
| R1-06-02 | 50 | 12 | 8 | 0 | 25.2 | 4.8 | 28.560 | 48 | h |
| R1-06-03 | 50 | 12 | 8 | 0 | 25.2 | 4.8 | 28.560 | 48 | h |
| R1-06-04 | 50 | 12 | 8 | 0 | 25.2 | 4.8 | 28.560 | 48 | h |
| R1-06-05 | 50 | 12 | 8 | 0 | 25.2 | 4.8 | 28.560 | 48 | h |
| R1-07-05 | 50 | 12 | 8 | 0 | 25.2 | 4.8 | 28.560 | 40 | h |
| R1-07-06 | 50 | 12 | 8 | 0 | 25.2 | 4.8 | 28.560 | 39 | h |
| R1-07-07 | 50 | 12 | 8 | 0 | 25.2 | 4.8 | 28.560 | 48 | h |
| R1-07-08 | 50 | 12 | 8 | 0 | 25.2 | 4.8 | 28.560 | 54 | h |
| R1-02-01 | 50 | 12 | 8 | 0 | 25.2 | 4.8 | 28.560 | 57 | h |
| R1-09-10 | 57 | 5 | 8 | 0 | 25.2 | 4.8 | 26.718 | 36.75 | h |
| R1-47-01 (bis) | 50 | 12 | 12 | 0 | 22 | 4 | 38.040 | 11 | h |
| R1-47-03 A (3.2) | 50 | 12 | 8 | 0 | 25.2 | 4.8 | 21.095 | 11 | h |
| R1-42-02 | 50 | 12 | 8 | 0 | 25.2 | 4.8 | 6.845 | 17 | d |
| R1-37-10 (A) | 50 | 8 | 8 | 0 | 29.2 | 4.8 | 4.703 | 11 | d |

| | | | | | | | | |
|---|---|---|---|---|---|---|---|---|
| VST 224 | 50% | 3% | 8% | 0 | 35% | 4% | 22.946 | 28 to 30 h |

### Medium-Life Bolus (31 Days to 90 Days)

Some examples of medium-life formulations according to the invention, in the form of a bolus, are given below.

TABLE 27
Ingredients of Medium-life bolus

| | | Accelerating | | | |
|---|---|---|---|---|---|
| Product | Active principle | agent | Delaying agent | Compression agent | Ballast |
| R1-37-09 | Cobalt carbonate | Arbo C12 | Hydrogenated | | Iron powder |
| (A) | | | soybean oil | | |
| R1-42-01 | Copper oxide | Arbo C12 | Hydrogenated | | Iron powder |
| | | | soybean oil | | |
| R2-47-02- | Mixture of PA: Glycinates of | Arbo C12 | Hydrogenated | Magnesium oxide | Iron powder |
| A(1.5) | copper 6.12%, of zinc 2.05%, | | soybean oil | | |
| | and of manganese 6.06%, | | | | |
| | Carbonates of copper 2.67% and | | | | |
| | of manganese 6.00%, zinc oxide | | | | |
| | 7.10%, Calcium iodate 1.56%, | | | | |
| | Sodium selenite 0.22%, beta- | | | | |
| | carotene 2.00%, vit. A 0.54%, | | | | |
| | vit. D3 0.05% and vit. E 3.8% | | | | |
| Mg/Cu/ | Mixture of macro and trace | Arbo C12 | Hydrogenated | Magnesium oxide | Iron powder |
| Se bolus | elements | | soybean oil | | |
| (test 3) | | | | | |
| R2-47-03 | Mixture of PA: Oxides of | Arbo C12 | Hydrogenated | Magnesium oxide | Iron powder |
| A(3.1) | copper 7.47%>, of zinc 23.60%, | | soybean oil | | |
| | Carbonates of manganese 13.27% | | | | |
| | and of cobalt 0.846%, Calcium | | | | |
| | iodate 3.34%, Sodium selenite | | | | |
| | 0.551% vit. A 0.713%, vit. E | | | | |
| | 14.75% | | | | |
| R1-18-01 | Copper carbonate | Arbo C12 | Hydrogenated | Tricalcium | Iron powder |
| | | | soybean oil | phosphate | |
| R1-16-09 | Copper carbonate | Arbo C12 | Hydrogenated | Slaked lime | Iron powder |
| | | | soybean oil | | |
| R1-32-04 | Copper carbonate | Arbo C12 | Hydrogenated | Quicklime | Iron powder |
| | | | soybean oil | | |
| R1-22-02 | Copper carbonate | Arbo C12 | Hydrogenated | CaCO3 production | Iron powder |
| | | | soybean oil | | |
| R1-22-08 | Copper carbonate | Arbo C12 | Hydrogenated | Heavy Mg | Iron powder |
| | | | soybean oil | carbonate | |
| R1-22-01 | Copper carbonate | Arbo C12 | Hydrogenated | Anhydrous | Iron powder |
| | | | soybean oil | dicalcium | |
| | | | | phosphate No. 1 | |
| R1-16-05 | Copper carbonate | Arbo C12 | Hydrogenated | Anhydrous | Iron powder |
| | | | soybean oil | dicalcium | |
| | | | | phosphate No. 2 | |
| R1-16-06 | Copper carbonate | Arbo C12 | Hydrogenated | Lithotamnion | Iron powder |
| | | | soybean oil | powder | |
| R1-16-07 | Copper carbonate | Arbo C12 | Hydrogenated | High-density | Iron powder |
| | | | soybean oil | heavy calcium | |
| | | | | carbonate | |
| R1-22-07 | Copper carbonate | Arbo C12 | Hydrogenated | Magnesium | Iron powder |
| | | | soybean oil | hydroxide | |
| R1-12-02 | Copper carbonate | Arbo C12 | Hydrogenated | Magnesium oxide | Iron powder |
| | | | soybean oil | | |
| R1-51-04 | Copper carbonate | Arbo C12 | Hydrogenated | Magnesium oxide | Iron powder |
| | | | soybean oil | | |
| R1-12-01 | Copper carbonate | Arbo C12 | Hydrogenated | Magnesium oxide | Iron powder |
| | | | soybean oil | | |
| Plant | Mixture of powders of | Arbo C12 | Hydrogenated | Magnesium oxide | Iron powder |
| bolus Test | medicinal plants (absinthe, | | colza oil | Calcium carbonate | |
| 11 4.7 | cucurbit, goosefoot, male | | | | |
| | fern, tansy, boldo, garlic, | | | | |
| | elecampane, artichoke, thyme) + | | | | |
| | lithothamnion | | | | |
| Plant | Mixture of powders of | Arbo C12 | Hydrogenated | Magnesium oxide | Iron powder |
| bolus Test | medicinal plants (absinthe, | | colza oil | Calcium carbonate | |
| 12 2.4 | cucurbit, goosefoot, male | | | | |
| | fern, tansy, boldo, garlic, | | | | |
| | elecampane, artichoke, thyme) + | | | | |
| | lithothamnion | | | | |

The percentages of the various ingredients, their solubility index, and their disintegration times are given below:

TABLE 28
Amounts of Ingredients, Medium-life bolus

| | | Accel. | Delaying | Comp. | Ballast | Excip. | Sol. | Time |
|---|---|---|---|---|---|---|---|---|
| Product | P.A % | Agent % | agent % | Agent, % | % | % | Index | (days) |
| R1-37-09 (A) | 50 | 4 | 8 | 0 | 33.2 | 4.8 | 2.562 | 32 |
| R1-42-01 | 50 | 12 | 8 | 0 | 25.2 | 4.8 | 2.562 | 70 |
| R2-47-02-A (1.5) | 38.17 | 8 | 5 | 19.03 | 25 | 4.8 | 11.107 | 40 |
| Mg/Cu/Se bolus | 19.13 | 7 | 1 | 62.37 | 7 | 3.5 | 10.974 | 45 |
| (Test 3) | | | | | | | | |
| R2-47-03 A (3.1) | 64.55 | 7 | 5 | 8.65 | 10 | 4.8 | 4.734 | 61 |
| R1-18-01 | 50 | 4 | 8 | 15 | 18.2 | 4.8 | 2.562 | 68 |
| R1-16-09 | 50 | 4 | 8 | 15 | 18.2 | 4.8 | 2.583 | 79 |
| R1-32-04 | 50 | 4 | 8 | 15 | 18.2 | 4.8 | 2.580 | 81 |
| R1-22-02 | 50 | 4 | 8 | 15 | 18.2 | 4.8 | 2.562 | 47 |
| R1-22-08 | 50 | 4 | 8 | 15 | 18.2 | 4.8 | 2.568 | 46 |
| R1-22-01 | 50 | 4 | 8 | 15 | 18.2 | 4.8 | 2.565 | 60 |
| R1-16-05 | 50 | 4 | 8 | 15 | 18.2 | 4.8 | 2.565 | 58 |
| R1-16-06 | 50 | 4 | 8 | 15 | 18.2 | 4.8 | 2.562 | 56 |
| R1-16-07 | 50 | 4 | 8 | 15 | 18.2 | 4.8 | 2.562 | 56 |
| R1-22-07 | 50 | 4 | 8 | 15 | 18.2 | 4.8 | 2.562 | 72 |
| R1-12-02 | 50 | 8 | 8 | 15 | 14.2 | 4.8 | 4.703 | 70 |
| R1-51-04 | 50 | 6 | 8 | 15 | 16.2 | 4.8 | 3.633 | 74 |
| R1-12-01 | 50 | 4 | 8 | 15 | 18.2 | 4.8 | 2.562 | 77 |
| Plant bolus Test | 35.7 | 3 | 5 | 9.0 (MgO) | 35 | 4.5 | | 85 |
| 11 4.7 | | | | 7.8 (CaCO3) | | | | |
| Plant bolus Test | 35.7 | 3 | 5 | 11.8 (MgO) | 35 | 4.5 | | 70 |
| 12 2.4 | | | | 5 (CaCO3) | | | | |

### Long-Life Bolus (91 Days to 180 Days)

Some examples of long-life formulations according to the invention, in the form of a bolus, are given below.

TABLE 29
Compositions of Long-life bolus

| | | Accel. | Delaying | Comp. | Ballast, | Excip. | Sol. | Time |
|---|---|---|---|---|---|---|---|---|
| Product | P.A % | Agent % | agent % | Agent, % | % | % | Index | (days) |
| R1-51-03 | 50 | 6 | 8 | 15 | 16.2 | 4.8 | 3.633 | 98 |
| | Copper oxide | Arbo C12 | Hydrogenated | Mg oxide | Iron | | | |
| | | | soybean oil | | powder | | | |
| R2-07-01 | 66.525 | 3 | 5 | 10.675 | 10 | 4.8 | 2.592 | 100 |
| | Mixture of carbonates | Arbo C12 | Hydrogenated | Magnesium | Iron | | | |
| | of Co 0.860%, Mn 13.5%, | | soybean oil | oxide | powder | | | |
| | oxides of Cu 7.6% and | | | | | | | |
| | Zn 24%, vitamins A 0.725% | | | | | | | |
| | and E 15.88%, Sodium | | | | | | | |
| | selenite 0.560%, and | | | | | | | |
| | Ca iodate 3.4% | | | | | | | |
| R1-16-14 | 62.57 | 4 | 5 | 13.63 | 10 | 4.8 | 3.128 | 101 |
| | Mixture of carbonates | Arbo C12 | Hydrogenated | Magnesium | Iron | | | |
| | of Co 0.860%, Mn 13.5%, | | soybean oil | oxide | powder | | | |
| | oxides of Cu 7.6% and | | | | | | | |
| | Zn 20%, vitamins A 0.722% | | | | | | | |
| | and E 15.88%, Sodium | | | | | | | |
| | selenite 0.560%, and | | | | | | | |
| | Ca iodate 3.4% | | | | | | | |
| R1-22-09 | Copper oxide: 41.6 | 4 | 5 | 6.8 | 12.3 | 4.875 | 3.009 | 160 |
| | Zinc oxide: 25, Sodium | Arbo C12 | Hydrogenated | Magnesium | iron | | | |
| | selenite: 0.445 | | soybean oil | oxide | powder | | | |
| R1-13-01 | 50 | 6 | 8 | 15 | 16.2 | 4.8 | 3.633 | 131 |
| | Manganese carbonate | Arbo C12 | Hydrogenated | Magnesium | iron | | | |
| | | | soybean oil | oxide | powder | | | |
| R1-16-15 | 61.8 | 4 | 5 | 15 | 9.285 | 4.875 | 2.797 | 176 |
| | Zinc oxide, 31; | Arbo C12 | Hydrogenated | Magnesium | iron | | | |
| | carbonates of copper, | | soybean oil | oxide | powder | | | |
| | 12.5; of cobalt, 1.52; | | | | | | | |
| | and of manganese, 15; | | | | | | | |
| | CaIO3, 1.6; Sodium | | | | | | | |
| | selenite, 0.22 | | | | | | | |
| R1-22-12 | Copper oxide: 11.6 | 4 | 5 | 6.78 | 12.3 | 4.875 | 3.009 | 138 |
| | Zinc oxide: 55, Sodium | Arbo C12 | Hydrogenated | Magnesium | iron | | | |
| | selenite: 0.445 | | soybean oil | oxide | powder | | | |
| VST228-1 | 62.88 | 3 | 7 | 11.32 | 12.3 | 3.5 | 3.229 | 145 |
| bis Test 2 | Black copper oxide 57.83%, | Arbo C12 | Hydrogenated | Magnesium | Iron | | | |
| 3.4 | Calcium iodate 3.95%, | | colza oil | oxide | powder | | | |
| | Sodium selenite 1.1% | | | | | | | |
| VST228-4 | 61.67 | 3 | 6 | 12.53 | 12.3 | 4.5 | 3.373 | 125 |
| bis Test 3 | Black copper oxide 57.83%, | Arbo C12 | Hydrogenated | Magnesium | Iron | | | |
| 3.4 | Calcium iodate 2.74%, | | colza oil | oxide | powder | | | |
| | Sodium selenite 1.1% | | | | | | | |
| VST 228-6 | 61.07%: | 3% | 5% | 13.76% | 12.68% | 4.5% | | 125 |
| 4.2 | Black copper oxide 52.05%, | Arbo C12 | Hydrogenated | Magnesium | Iron | | | |
| | Calcium iodate 7%, | | colza oil | oxide | powder | | | |
| | Sodium selenite 2.02% | | | | | | | |
| VST 215 | 66.13%: | 3% | 3% | 11.04% | 12% | 4.83% | 2.766 | 180 |
| D12H1 | Mixture of vitamin E 20% | Arbo C12 | Hydrogenated | Magnesium | Iron | | | |
| | and of vitamin A 2%, | | colza oil | oxide | powder | | | |
| | Oxides of manganese | | | | | | | |
| | 8.684%, of copper 7.6% | | | | | | | |
| | and of zinc 21.725%, | | | | | | | |
| | Cobalt carbonate 0.86%, | | | | | | | |
| | Calcium iodate 4.53%, | | | | | | | |
| | Sodium selenite 0.732% | | | | | | | |

Ultralong-Life Bolus (More than 180 Days)

Some examples of ultralong-life formulations according to the invention, in the form of a bolus, are given below.

TABLE 30
Compositions of Ultralong-life Bolus

| | | Accel. | Delaying | Comp. | Ballast, | Excip. | Sol. | Time |
|---|---|---|---|---|---|---|---|---|
| Product | P.A % | Agent % | agent % | Agent, % | % | % | Index | (days) |
| R1-26-04 | 50 | 4 | 8 | 15 | 18.2 | 4.8 | 2.583 | 215 |
| | Zinc oxide | Arbo C12 | Hydrogenated | Slaked lime | Iron | | | |
| | | | soybean oil | | powder | | | |
| R1-22-10 | Copper oxide: 31.6 | 4 | 5 | 6.78 | 12.3 | 4.875 | 3.009 | 200 |
| | Zinc oxide: 35, | Arbo C12 | Hydrogenated | Magnesium | Iron | | | |
| | Sodium selenite: 0.445 | | soybean oil | oxide | powder | | | |
| R1-22-11 | Copper oxide: 21.6 | 4 | 5 | 6.78 | 12.3 | 4.875 | 3.009 | 200 |
| | Zinc oxide: 45, | Arbo C12 | Hydrogenated | Magnesium | Iron | | | |
| | Sodium selenite: 0.445 | | soybean oil | oxide | powder | | | |
| R1-12-05 | Zinc oxide: 50 | 6 | 8 | 15 | 16.2 | 4.8 | 3.633 | 265 |
| | | Arbo C12 | Hydrogenated | Magnesium | Iron | | | |
| | | | soybean oil | oxide | powder | | | |
| VST 226-6 | 68.12%: | 3% | 5% | 6.78% | 12.6% | 4.5% | 3.317 | 300 |
| 2R | Mixture of oxides | Arbo C12 | Hydrogenated | Magnesium | Iron | | | |
| | of Copper 21.99% | | colza oil | oxide | powder | | | |
| | and of Zinc 38.61%, | | | | | | | |
| | Cobalt carbonate 1.28%, | | | | | | | |
| | Sodium selenite 1.03%, | | | | | | | |
| | Calcium iodate 5.21% | | | | | | | |
| VST 227-4 | 64.65%: | 3% | 6% | 9.55% | 12.3% | 4.5% | 3.359 | 275 |
| Test 3 | Mixture of oxides | Arbo C12 | Hydrogenated | Magnesium | Iron | | | |
| 4.2 | of Copper 23.05% | | colza oil | oxide | powder | | | |
| | and of zinc 40.5%, | | | | | | | |
| | Sodium selenite 1.1% | | | | | | | |

1-75. (canceled)

76. A galenical composition suitable for administration to an animal, said composition comprising at least the following three components:
one or more active principles;
a disintegration accelerating agent, which is a lignosulfonate in water soluble powdered form;
a disintegration delaying agent, which is a fat that is solid at room temperature;
and in which the disintegration accelerating agent and the disintegration delaying agent are integrated in the composition so as to form a matrix with controlled release of the active principle or principles.

77. The galenical composition as claimed in claim 76, wherein the controlled-release matrix is formed by homogeneous mixing of the active principle or principles with the disintegration delaying agent and the disintegration accelerating agent.

78. The galenical composition as claimed in claim 76, which further comprises a ballasting agent which is a metal or metal alloy in particulate form.

79. The composition as claimed in claim 76, wherein the composition is in the form of a bolus.

80. The composition as claimed in claim 76, wherein the composition is in the form of tablets to be crunched.

81. The composition as claimed in claim 76, wherein the disintegration accelerating agent is present in amounts between 3 wt % and 25 wt % relative to the total weight of the composition.

82. The composition as claimed in claim 76, wherein the delaying agent is present in amounts between 1% and 12% relative to the total weight of the composition.

83. The composition as claimed in claim 76, displaying complete disintegration between one hour and less than or equal to 30 days.

84. The composition as claimed in claim 76, displaying complete disintegration greater than 30 days and less than or equal to 90 days.

85. The composition as claimed in in claim 76, displaying complete disintegration greater than 90 days and less than or equal to 180 days.

86. The composition as claimed in claim 76, displaying complete disintegration greater than 180 days.

87. The composition as claimed in claim 76, which further comprises a compression agent of mineral origin based on calcium, magnesium or phosphorus being in the form of an inorganic salt of a mineral nature, or a mixture thereof, in powdered form and is selected from the group consisting of one or more salts based on calcium, magnesium, or phosphorus, having a solubility in water at room temperature below 0.25 g in 100 g of water.

88. The composition as claimed in claim 87, wherein the compression agent is present in the composition at values between 0% and 70% relative to the total weight of the composition.

89. The composition as claimed in claim 76, wherein the active principle or principles is a trace element or a mixture of trace elements, a vitamin or a mixture of vitamins, a macroelement or a mixture of macroelements based on calcium, magnesium, phosphorus, potassium, sodium or sulfur having a solubility in water above 0.25 g in 100 g of water, a prebiotic or a mixture of prebiotics, a probiotic or a mixture of probiotics, an amino acid, a peptide, a protein, an enzyme or a mixture thereof, a vegetable extract, a fraction or a purified molecule of the latter, an essential oil, a purified aromatic molecule, or a mixture thereof, all in the form of a powder, a molecule with a therapeutic use or a mixture thereof.

90. The composition as claimed in claim 89, wherein the amino acid, peptide, protein, enzyme or mixture thereof is selected from the group consisting of proteins or water-soluble concentrates of milk proteins in powder form, lyophilized or atomized colostrum powder, whey in the form of powder, purified or IgG-enriched fractions, lactoferrin, lactoperoxidase, animal or vegetable enzymes, Promutase, 3-phytase, 6-phytase, endo-1,4-betaglucanases, endo-1,4-betaxylanases, enzymes that improve or promote an animal's digestion, free amino acids or in the form of salts or peptides, L-carnitine, or any other water-soluble peptide of molecular weight greater than a dipeptide.

91. The composition as claimed in claim 76, which further comprises a ballasting agent in the form of particulate iron in amounts between 0% and 50% relative to the total weight of the composition.

92. A method to stimulate milk production in a nonhuman female animal, said method comprising administering an effective amount of calcium and/or magnesium pidolate to said animal.

93. The method as claimed in claim 92, wherein the animal is a ruminant selected from the group consisting of bovines, sheep, goats, the deer family, the camel family.

94. The method as claimed in claim 92, wherein the nonhuman animal is monogastric selected from the group consisting of pigs, the rabbit family, equines, pets.

95. The method as claimed in claim 92, wherein the calcium and/or magnesium pidolate is integrated as active principle in a galenical composition as claimed in claim 76.

96. A method for supplying trace elements for a nonhuman animal, said method comprising administering to said animal the composition of claim 76.

97. The method as claimed in claim 96, wherein the nonhuman animal is a ruminant and the composition is in the form of a bolus, or the nonhuman animal is monogastric and the composition is in the form of tablets to be crunched.

98. The method as claimed in claim 96, wherein the nonhuman animal is selected from the group consisting of bovines, sheep, goats, the deer family, the camel family, pigs, the rabbit family, equines, and pets.

99. A method for stimulating, at around the time of parturition, general mobilization of calcium ions in a nonhuman female mammal, said method comprising administering to said mammal an effective amount of the composition of claim 76.

100. The method as claimed in claim 99, wherein the composition is in the form of a bolus or in tablet form.

101. A method for increasing the weight gain of a suckling piglet, said method comprising administering to said piglet an effective amount of the composition of claim 76.

102. A method for speeding up farrowing time of an animal, said method comprising administering to said animal an effective amount of the composition of claim 76.

103. A method for maintaining blood calcium level above 85 mg/l peri-partum in a cow, said method comprising administering to said cow at least 3.78 g of calcium in the form of pidolate salt.

104. A method for increasing a blood phosphorus level above 55 mg/l peri-partum in a cow, said method comprising administering to said cow at least 30.24 g of calcium pidolate and/or 7.5 g of magnesium.
